(19) United States
(12) Patent Application Publication
(10) Pub. No.: US 2026/0306024 A1
(43) Pub. Date: Oct. 8, 2026

(54) BRAIN-COMPUTER INTERFACE (BCI) SYSTEMS WITH MAGNETIC ARRANGEMENT FOR ALIGNMENT

(71) Applicant: Echo Neurotechnologies Corporation, San Francisco, CA (US)

(72) Inventors: Jason Ern-Chi Chung, San Francisco, CA (US); Ryan Thomas Bauer, Maple Grove, MN (US); Michael Muir Allen, Orinda, CA (US); Kedar Gautam Shah, San Francisco, CA (US); Michael C. Holzbaur, Mountain View, CA (US); Pierre Guy Vancaillie, Sydney (AU)

(21) Appl. No.: 19/638,524

(22) Filed: Apr. 3, 2026

Related U.S. Application Data
(60) Provisional application No. 63/783,858, filed on Apr. 4, 2025.

(51) Int. Cl. A61B 5/00; G06F 3/01

(52) U.S. Cl. CPC A61B 5/686; A61B 2560/0219; A61B 2562/225; G06F 3/015

(57) ABSTRACT

A cranial implant device comprises a frame that is configured to be anchored to a skull of a patient. The frame includes one or more channels positioned along one or more sides of the frame. The one or more channels are configured to hold a plurality of first magnets. The frame includes a mating area configured to mate with a hermetically sealed package. The hermetically sealed package is configured to be inserted through an opening in the skull and positioned in the mating area.

### RELATED APPLICATIONS

This application claims priority to U.S. Provisional Application No. 63/783,858, filed Apr. 4, 2025, which is incorporated by reference herein in its entirety.

### TECHNICAL FIELD

The disclosed embodiments relate generally to brain computer interface (BCI) systems, and more specifically to implant devices that include magnets for coupling and aligning with external devices.

### BACKGROUND

Neurological conditions can result in the loss of communication and autonomy. Brain computer interface (BCI) technologies can assist individuals with neurological impairments, by recording and processing brain signals and translating the signals into outputs.

### SUMMARY

The present disclosure describes, among others, a BCI system that includes an implant device having magnets for aligning the implant device to an external wearable device. In accordance with some embodiments, the implant device is configured to couple to the wearable device and transmit neural signals wirelessly to the wearable device. To achieve faster, more accurate, and more natural communication, the BCI system can include a high-density electrode array with at least 1000 electrode channel counts, meaning that the implant device has to transmit the at least 1000 electrode channels of neural data, in-real time, to the wearable device. In order to achieve these performance requirements, and ensure efficient data communication and powering, alignment of the implant device (e.g., the antennas) with the external device is critical.

The present disclosure describes, amongst others, an implant device that includes a frame that is configured to be anchored to a skull of a patient. The frame includes one or more channels (e.g., slots) positioned along at least a part of the perimeter of the frame (e.g., on one or more sides of the frame). The channels are configured to hold one or more magnets within the channels. In some embodiments, the one or more magnets can be securely attached within one or more hermetically sealed packages. The hermetically sealed packages are then placed inside the one or more channels.

In accordance with some embodiments, a cranial implant device includes a frame that is configured to be anchored to a skull of a patient. The frame includes one or more channels positioned along one or more sides of the frame. The one or more channels are configured to hold a plurality of first magnets. The frame includes a mating area configured to mate with a hermetically sealed package. The hermetically sealed package is configured to be inserted through an opening in the skull and positioned in the mating area.

In some embodiments, adjacent magnets of the plurality of first magnets have opposite polarities.

In some embodiments, the plurality of first magnets are configured to magnetically couple the frame to an external device that is positioned external to the patient.

In some embodiments, the external device includes a plurality of complementary magnets. The plurality of first magnets are configured to magnetically couple to the external device via the plurality of complementary magnets.

In some embodiments, the cranial implant device includes a set of electronic components enclosed in the hermetically sealed package.

In some embodiments, the cranial implant device does not include a battery.

In accordance with some embodiments, a brain computer interface (BCI) system includes a cranial implant device and an external device, where the external device configured to provide constant power to the cranial implant device.

Thus methods, devices, and systems are disclosed that align an implant device with an external device.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

For a better understanding of the aforementioned systems, methods, and graphical user interfaces, reference should be made to the Detailed Description of Embodiments below, in conjunction with the following drawings in which like reference numerals refer to corresponding parts throughout the figures. The accompanying drawings, which are included to provide a further understanding of the embodiments, are incorporated herein, constitute a part of the specification, illustrate the described embodiments, and, together with the description, serve to explain the underlying principles.

FIG. 1A illustrates an example operating environment, in accordance with some embodiments.

FIG. 1B illustrates example operating modes of a computing device, in accordance with some embodiments.

FIG. 1C is a graph showing an actual time-series dataset of neural activity of a patient, in accordance with some embodiments.

FIG. 1D is a graph showing neural features extracted from the neural activity dataset of FIG. 1C, in accordance with some embodiments.

FIGS. 2A, 2B, and 2C illustrate an example brain implant device, in accordance with some embodiments.

FIG. 2D is a block diagram showing an example data path, control path, and power path of a brain implant device, in accordance with some embodiments.

FIG. 3A illustrates an example wearable device, in accordance with some embodiments.

FIG. 3B is a block diagram showing an example data path, control path, and power path of a wearable device, in accordance with some embodiments.

FIG. 4 is a block diagram illustrating an example computing device in accordance with some embodiments.

FIG. 5 is a block diagram illustrating an example server system in accordance with some embodiments.

FIGS. 6A, 6B-1, 6B-2, 6B-3, 6C, and 6D illustrate various views of an implant device, in accordance with some embodiments.

FIGS. 7A and 7B illustrate an implant device, in accordance with some embodiments.

FIGS. 8A, 8B, and 8C illustrate an implant device, in accordance with some embodiments.

FIGS. 9A and 9B illustrate an implant device, in accordance with some embodiments.

FIGS. 10A and 10B illustrate an implant device, in accordance with some embodiments.

FIGS. 11A and 11B illustrate an implant device, in accordance with some embodiments.

FIGS. 12A and 12B illustrate an implant device, in accordance with some embodiments.

FIG. 13A illustrates a side view of a patient, in accordance with some embodiments.

FIG. 13B is a cross-sectional view depicting insertion of the implant device into the skull of a human subject, in accordance with some embodiments.

FIGS. 14A, 14B, 14C, and 14D illustrate a patch component and an implant device, in accordance with some embodiments.

FIGS. 15A, 15B, and 15C illustrate perspective and exploded views of an implant device, in accordance with some embodiments.

FIGS. 16A to 16E illustrate exemplary frames to of an implant device, in accordance with some embodiments.

FIG. 17 illustrates an implant device, in accordance with some embodiments.

FIGS. 18A to 18C illustrate a patch component, in accordance with some embodiments.

Like reference numerals refer to corresponding parts throughout the several views of the drawings.

### DETAILED DESCRIPTION OF EMBODIMENTS

Reference will now be made in detail to specific embodiments, examples of which are illustrated in the accompanying drawings. In the following detailed description, numerous non-limiting specific details are set forth in order to assist in understanding the subject matter presented herein. But it will be apparent to one of ordinary skill in the art that various alternatives may be used without departing from the scope of the claims and the subject matter may be practiced without these specific details. For example, it will be apparent to one of ordinary skill in the art that the subject matter presented herein can be implemented on many types of electronic devices with digital video capabilities.

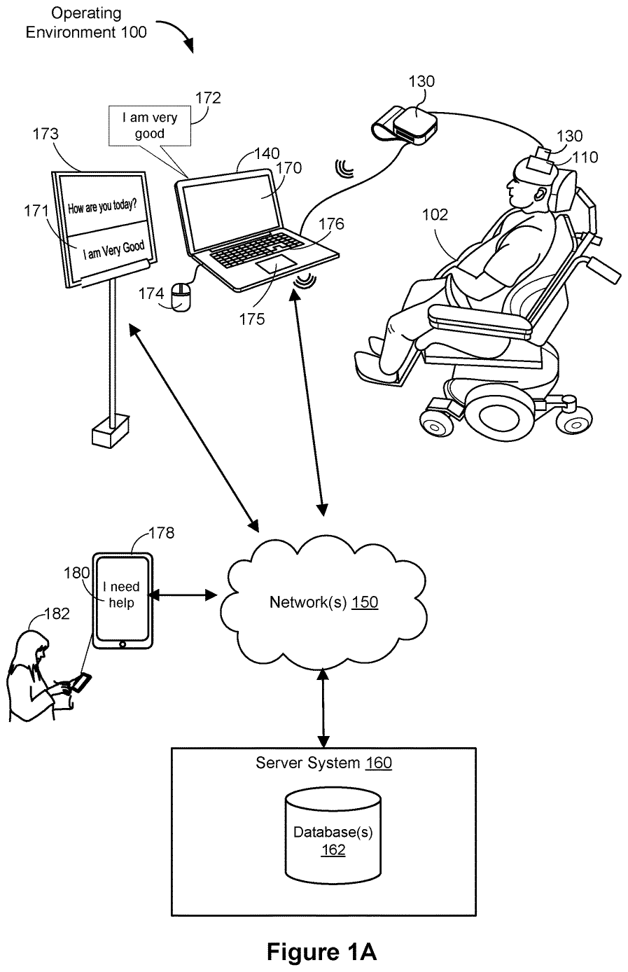

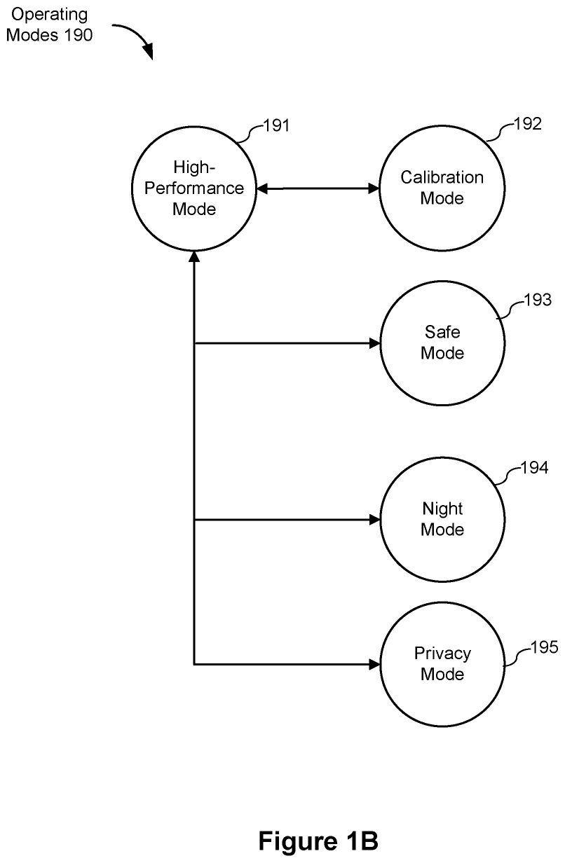

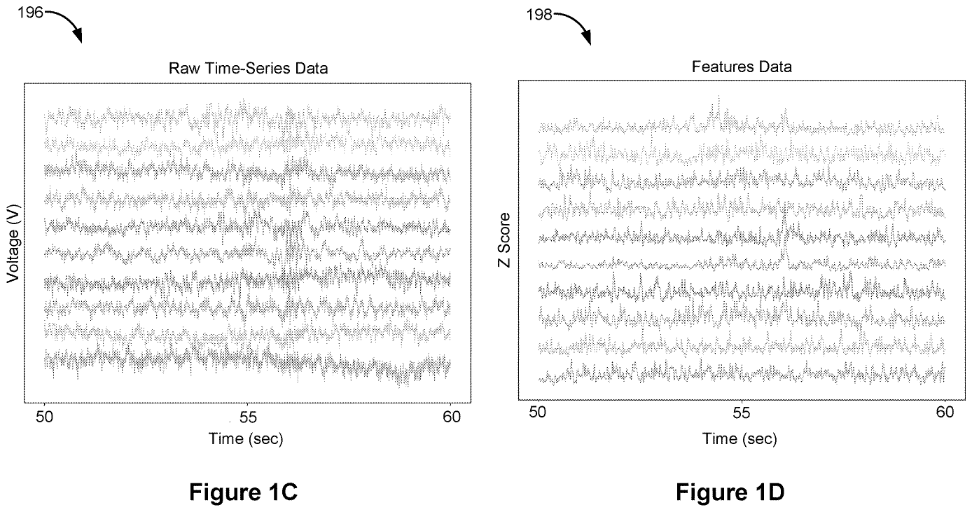

FIG. 1A illustrates an example operating environment 100 of a brain computer interface (BCI) system, in accordance with some embodiments.

As used herein, a BCI system is a system that acquires brain signals from a user and determines, from the signals, into one or more intended actions (e.g., functional intents) of the user. In some embodiments, the BCI system generates and executes commands for controlling one or more external devices without requiring traditional muscle movements of the user. Because no user movement or vocalization is required to control the BCI system, the BCI system can assist users with disabilities, thereby enhancing the user's capabilities and improving their quality of life.

The operating environment 100 depicts a human subject 102. As used herein, human subject 102 is also referred to as a “patient” or a “user.” In some instances, the human subject 102 is unable to speak or unable to move their limbs. In some embodiments, the BCI system includes an implant device 110 (also referred to herein as a cranial implant device), which is implanted into the skull of the human subject 102. The implant device 110 includes an array of high-density electrode sensors for recording neural signals (e.g., electrical signals) corresponding to motor cortex activity directly from the brain surface of the human subject 102. In some embodiments, the array is a brain-penetrating electrode array or an electrocorticography (ECoG) electrode array. In some embodiments, the electrodes are depth electrodes or surface electrodes. In some embodiments, the recorded neural signals represent specific activities of the brain, including attempted speech, attempted movement, hearing, and/or vision. In some embodiments, the array of electrode sensors is placed on the cortical surface of the brain, either epidurally or subdurally, to record signals directly from the brain. In some embodiments, the array of electrode sensors can penetrate under the scalp.

In some embodiments, the BCI system includes a wearable device 130. As described in further detail in FIG. 3A, in some embodiments the wearable device 130 includes a patch component that is magnetically and communicatively coupled to the implant device 110. The patch component is configured to transmit power wirelessly into the implant device 110 and receive neural signal data from the implant device 110. In some embodiments, the wearable device 130 includes a relay component that includes electronics for supporting the functionalities of the patch component.

In some embodiments, the BCI system includes a computing device 140 that is communicatively connected with the wearable device 130. The computing device 140 can include a display 170, a keyboard 176 and a pointing device such as a mouse 174 or a touchpad 175. In accordance with some embodiments, the computing device 140 functions as the main interface for the human subject 102. For example, the human subject 102 can access one or more applications executing on the computing device 140 for their daily tasks and activities such as checking email and browsing the web. In some embodiments, the one or more applications executing on the computing device 140 includes a BCI application (e.g., application 440) that is configured to decode the neural signals and interpret the decoded signals to determine one or more intended actions (e.g., functional intents) of the human subject 102. In some embodiments, the BCI application applies one or more machine learning models that are stored locally on the computing device 140 to perform the decoding and interpreting. In some embodiments, the BCI application is configured to execute on the computing device 140 even while the computing device 140 is offline (although an internet connection will be required to receive new models from the server system 160).

In some embodiments, the BCI system includes a server system 160 that is communicatively coupled to the computing device 140 through one or more communication networks 150. The server system 160 includes an operational pipeline that is configured to automatically generate different machine learning models based on respective input data, train the models to optimize their performance on specific tasks, and deploy the models locally on respective computing devices 140. In some embodiments, the server system 160 includes one or more databases 162 for storing data such as raw data obtained from implanted devices corresponding to respective patients, feature data, analytics data, application information, and firmware versions.

With continued reference to FIG. 1A, in some embodiments, the BCI system is configured to control one or more output devices to carry out one or more intended actions of the human subject 102 according to the decoded signals. As one example, in some embodiments, the BCI system (e.g., computing device 140 or server system 160) is configured to control a keyboard (e.g., physical keyboard 176 or a virtual keyboard) to generate language and/or numeral outputs according to the decoded signals (e.g., by typing letters, numbers, words and/or sentences using the keyboard), without requiring any voice command and/or limb movement by the human subject 102. As another example, in some embodiments, the BCI system is configured to control a cursor (e.g., by controlling a pointing device such as mouse 174, touchpad 175, or the cursor) according to the decoded signals, without requiring any voice command and/or limb movement by the human subject 102. As another example, in some embodiments, the BCI system is configured to control a display device (e.g., display 170 or external display device 173) to display a visual output (e.g., text entry 171) corresponding to a response from the human subject 102, without requiring any voice command and/or limb movement by the human subject 102. As another example, in some embodiments, the BCI system is configured to translate a visual/text output into a verbal expression and generate an audio output 172 articulating the verbal expression using an audio output component of the computing device 140, without requiring any voice command and/or limb movement by the human subject 102. As another example, in some embodiments, the BCI system is configured to control one or more interfaces of the computing device 140 to navigate through menus and/or switch between tabs of one or more applications executing on the computing device 140, without requiring any voice command and/or limb movement by the human subject 102. As yet another example, in some embodiments, the BCI system is configured to generate an alert according to the decoded signals and transmit the alert to an electronic device 178 that is associated with a caregiver 182 of the human subject 102, for display (e.g., as alert message 180) on a display screen of the electronic device 178.

FIG. 1B illustrates example operating modes 190 of a computing device 140, in accordance with some embodiments.

In accordance with some embodiments, the computing device 140 can switch between various operating modes. A respective operating mode may have its associated (e.g., distinct) user interface. A human subject 102 may interact with the computing device 140 in specific ways, depending on the operating mode.

Referring to FIG. 1B, in some embodiments, the operating modes include a high performance mode 191 that allows a human subject 102 to fully interact with the computing device 140. For example, in the high performance mode 191, the human subject 102 has access to all functionalities of applications that are executing on the computing device. In some embodiments, the high performance mode 191 is also referred as the “normal” mode. In some embodiments, high performance mode 191 is the default operating mode of the computing device.

In some embodiments, the operating modes include a calibration mode 192, which is a sandbox mode used during training tasks.

In some embodiments, the operating modes include a safe mode 193. In some embodiments, the safe mode 193 is a fallback mode for when the high performance mode 191 is not available.

In some embodiments, the operating modes include a night mode 194. When operating in the night mode, the display screen 170 of the computing device 140 is not in use and the computing device 140 has limited functionality due to the screen not being in use.

In some embodiments, the operating modes include a privacy mode 195, where brain signals are not recorded and not decoded.

FIG. 1C is a graph 196 showing an actual raw time-series dataset of neural activity of a patient collected by an electrode array, in accordance with some embodiments. The dataset is collected by placing electrodes on the surface of the patient's brain. Each of the line plots in the graph 196 corresponds to electrical field signals collected by a respective channel (e.g., an electrode) of the array. The line plots on the graph 196 have been offset for clarity.

FIG. 1D is a graph 198 showing a neural-feature time-series dataset extracted from the neural activity data of FIG. 1C, in accordance with some embodiments. In some embodiments, the neural-feature time series dataset is generated using a feature processing pipeline that is executed by the computing device 140 (e.g., application 440) or the server system 160 (e.g., operational pipeline 524). The neural features correspond to representations of brain signals that contain information related to underlying neural processing, including neural representations associated with attempted speech and/or motor movements. The values on the y-axis of the graph 198 are z-score values, which are normalized values that indicate how many standard deviations a data point is from the mean of the dataset (computed individually for each feature time series).

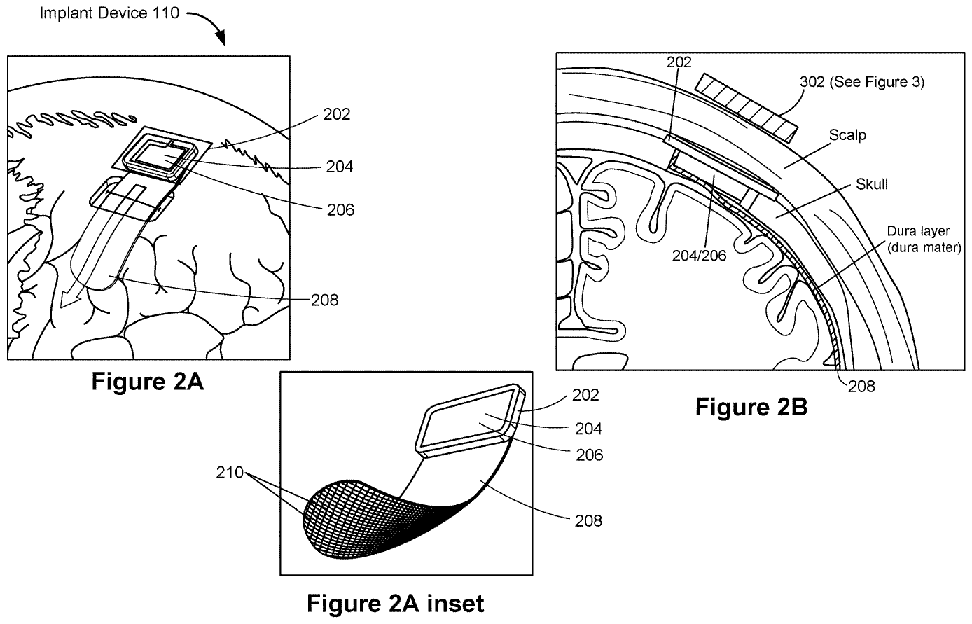

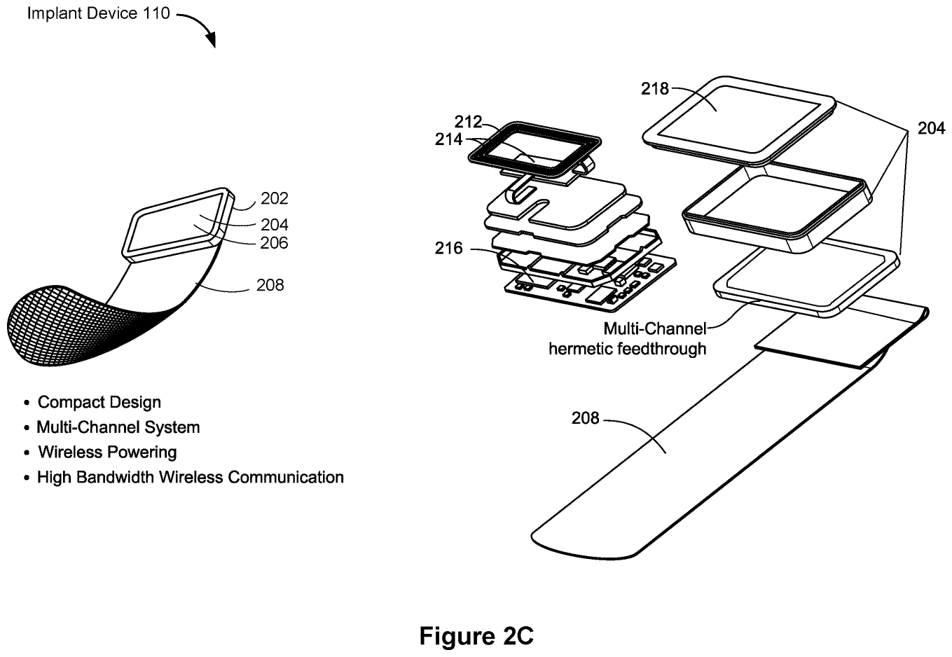

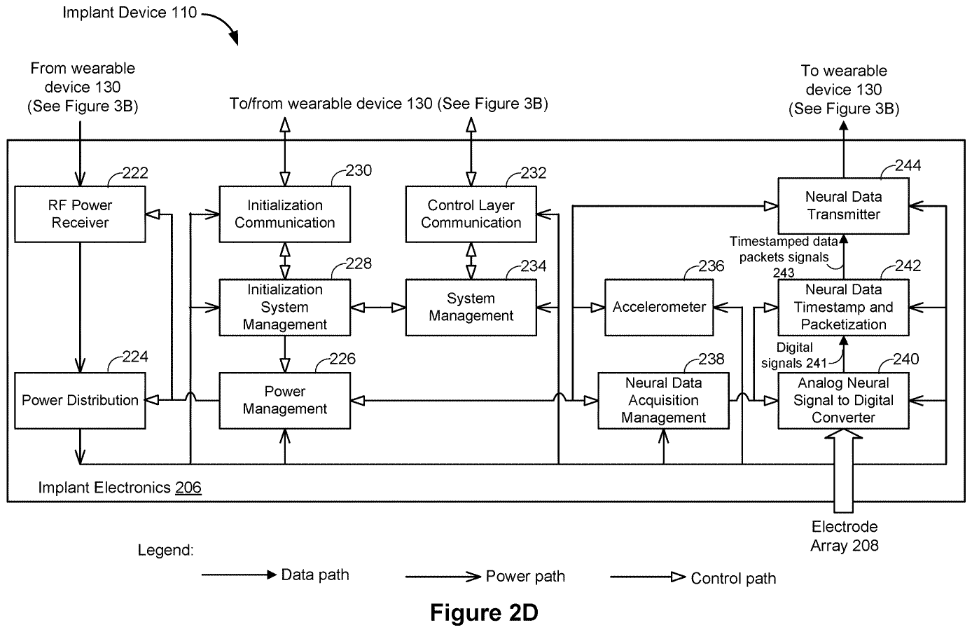

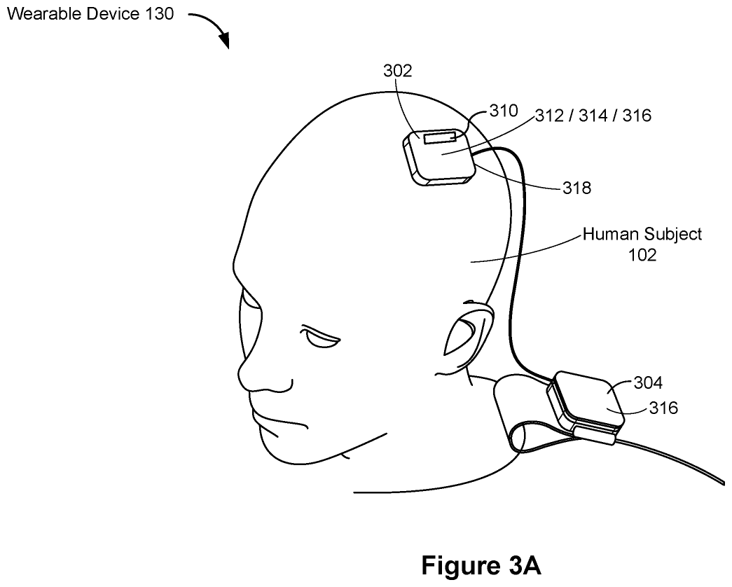

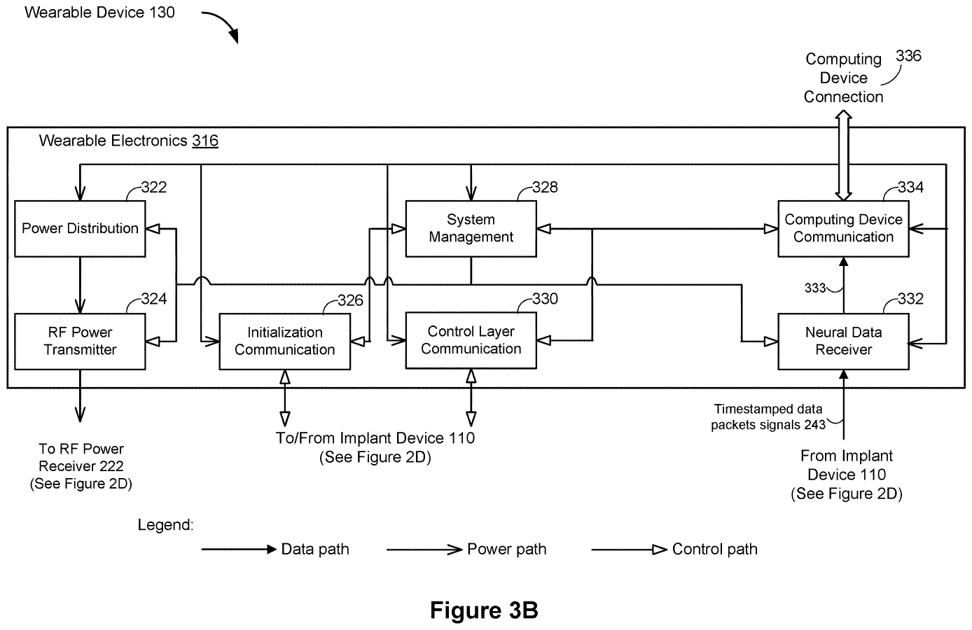

FIGS. 2A, 2B, and 2C illustrate various views of the implant device 110, in accordance with some embodiments.

In some embodiments, the implant device 110 includes a frame 202, a hermetic package 204 (e.g., hermetically-sealed package), implant electronics 206 (e.g., one or more electronic components) enclosed in the hermetic package 204, and an electrode array 208. As used herein, the electrode array 208 is also referred to as “an array of electrodes” or “an array of electrode sensors.” FIG. 2A inset shows that the electrode array 208 comprises a thin, flexible array (e.g., with a thickness of around 50 μm to 200 μm) that includes multiple electrode sensors 210. In some embodiments, the electrode array 208 comprises an electrocorticography (ECoG) array that is surgically implanted on the brain's surface.

In some embodiments, the electrode array 208 includes at least 100 electrodes, at least 250 electrodes, at least 500 electrodes, at least 750 electrodes, at least 1000 electrodes, at least 1500 electrodes, at least 2000 electrodes, at least 2500 electrodes, at least 3000 electrodes, at least 4000 electrodes, at least 5000 electrodes, at least 7500 electrodes, or at least 10,000 electrodes. In some embodiments, the electrode array 208 is designed to be placed over a substantial portion of or the entire sensorimotor cortex region, to collect electrophysiologic data with information content of intended speech and movement of a human subject. In some embodiments, the electrode array is designed to be placed over one or more subregions or regions of the cortex, such as the precentral gyrus, postcentral gyrus, posterior superior frontal gyrus, posterior middle frontal gyrus, posterior inferior frontal gyrus region, superior temporal gyrus, or a combination of any of the foregoing. Typically, each electrode 210 of the electrode array 208 corresponds to a channel and is configured to pick up electrical activity from the cortex. A denser electrode array with higher channel count can capture more signals with higher fidelity, which can be used to decode intended actions (e.g., intended speech actions and non-speech actions) of the human subject.

FIG. 2B illustrates a coronal cross-sectional view of a brain of a human subject. In some embodiments, the frame 202 is configured to be anchored to a skull of a human subject 102 via a surgical procedure called craniectomy, in which a scalp incision is made and a full-thickness section of bone is removed from the skull. In the example of FIG. 2B, the frame 202, the hermetic package 204, and the implant electronics 206 are configured to be positioned above the dural layer (e.g., closer to the scalp), whereas the electrode array 208 is inserted into the sub-dural layer (e.g., underneath the dura mater) of the brain of the human subject 102. During device operation, the electrode array 208 is configured to acquire (e.g., record) motor cortex neural signals (e.g., electrical signals) from the brain of the human subject.

In some embodiments, the implant device 110 includes a battery. In some embodiments, the implant device 110 does not include a battery. In some embodiments, the implant device 110 is configured to receive power signals from a patch component 302 of the wearable device 130. In some embodiments, the implant electronics 206 include a wireless powering coil 212 that is configured to receive electrical power wirelessly from the patch component 302. In some embodiments, the hermetic package 204 includes a cover 218 that is composed of a ceramic material or other dielectric material to facilitate the receiving of power signals via inductive coupling or capacitive coupling. In some embodiments, the wireless powering coil 212 is configured to receive constant electrical power from the patch component 302.

FIG. 2B illustrates coupling between the implant device 110 and the patch component 302. In some embodiments, the frame 202 includes one or more magnets that are configured to magnetically couple to one or more complementary magnets positioned at the patch component 302.

In some embodiments, the implant electronics 206 include a wireless communication antenna 214 that is configured to establish a direct wireless link between the implant device 110 and the wearable device 130. In some embodiments, the wireless communication antenna 214 is configured to implement high bandwidth wireless communication to transmit the multiple channels (e.g., hundreds or thousands of channels) of brain signals (e.g., electrical signals) that are collected by the electrode array 208 to the wearable device 130. In some embodiments, the implant device 110 is configured to amplify, digitize, and transmit the signals to the patch component 302 as data packets without interpreting and/or further processing the signals.

In some embodiments, the implant electronics 206 include processing circuitry, communication circuitry, and power circuitry. In some embodiments, the various circuitries are implemented as a multi-channel application-specific integrated circuit (ASIC) chip 216 that enables custom programming of specific tasks. In some embodiments, the ASIC chip 216 is implemented as a system-on-chip (SOC). In some embodiments, the ASIC chip 216 and includes one or more microprocessors and one or more memory blocks such as ROM, RAM, EEPROM, flash memory, or other building blocks.

FIG. 2D is a block diagram showing an example data path (represented by closed triangle arrows), power path (represented by two-edged arrows), and control path (represented by open triangle arrows) of an implant device 110, in accordance with some embodiments.

The data path of the implant device 110 includes receiving neural signals from the electrode array 208. In some embodiments, the implant device 110 includes an analog neural signal to digital converter module 240 (e.g., an analog to digital converter) for converting the neural signals (e.g., electrical signals) from analog form to digital form (e.g., digital signals 241). The implant device 110 includes a neural data timestamp and packetization module 242 for embedding date/time information to the digital signals and breaking down the digital signals into data packets (e.g., data segments) of smaller data sizes (e.g., timestamped data packets 243). The implant device 110 includes a neural data transmitter module 244 for transmitting the timestamped data packets 243 to the wearable device 130.

In accordance with some embodiments, the implant device 110 does not include a battery (i.e., the implant device 110 is not a battery-operated device). In some embodiments, the implant device 110 receives its power supply from the wearable device 130. In some embodiments, the implant device 110 includes an RF power receiver module 222 for receiving power (e.g., DC power) from the wearable device 130 (e.g., from RF power transmitter module 324 of the wearable device 130) and sending the received power to a power distribution module 224. The power distribution module 224 is configured to distribute the power to the various modules of the implant device 110, including the analog neural signal to digital converter module 240, the neural data timestamp and packetization module 242, the neural data transmitter module 244, a power management module 226, an initialization system management module 228, an initialization communication module 230, a control layer communication module 232, a system management module 234, an accelerometer module 236, and a neural data acquisition module 238.

In some embodiments, the power management module 226 is configured to actively control and monitor the flow of electrical power within the implant device 110. For example, the power management module 226 can regulate voltage, manage current draw, and protect modules such as the neural data acquisition module 238, the analog neural signal to digital converter module 240, the neural data timestamp and packetization module 242, the neural data transmitter module 244, and the accelerometer module 236 against overloads, thus ensuring efficient and safe power distribution to modules or components of the implant device 110.

In some embodiments, the initialization system management module 228 is configured to handle the initialization process when the implant device 110 first starts up, by coordinating the loading and configuration of settings and modules, such as the initialization communication module 230, the system management module 234, and the power management module 226.

In some embodiments, the initialization communication module 230 is configured to set up and prepare the communication channels and protocols needed for the implant device 110 to interact with the wearable device 130 at the beginning of its operation.

In some embodiments, the control layer communication module 232 is configured to manage the flow of data between different modules of the implant device 110 and between the implant device 110 and the wearable device 130.

In some embodiments, the system management module 234 is configured to manage and monitor the performance, health, and operation of the implant device 110. For example, the system management module 234 can track system parameters such as temperature, voltage, and power consumption to ensure stable operation of the implant device 110. In some instances, the system management module 234 can identify system failures, log errors, and provide alerts for troubleshooting.

In some embodiments, the accelerometer module 236 (e.g., hardware accelerator) is specifically designed to significantly speed up the execution of specific tasks by offloading them from the main processor of the ASIC chip 216.

In some embodiments, the neural data acquisition module 238 is configured to record electrical activity from the brain (e.g., neurons) using electrodes 210 of the electrode array 208. In some embodiments, the neural data acquisition module 238 is configured to acquire the electrical activity without performing additional processing on the recorded data. In some embodiments, the neural data acquisition module 238 is configured to boost weak neural signals. In some embodiments, the neural data acquisition module 238 is configured to filter out noise and unwanted frequencies to improve signal quality.

FIG. 3A illustrates a wearable device 130, in accordance with some embodiments. In some embodiments, the wearable device 130 includes a patch component 302, a relay component 304, and wearable electronics 316 (e.g., electronic components).

In some embodiments, the patch component 302 includes one or more magnets 310 that are configured to magnetically couple and align the patch component 302 to the implant device 110. In some embodiments, the patch component 302 includes one or more coils 312 and/or antenna 314 for transmitting power wirelessly into the implant device 110, transmitting command signals into the implant device 110, and/or receiving neural signal data transmitted from the implant device 110. In some embodiments, the patch component 302 includes wearable electronics 316 (e.g., electronic components) (e.g., to power the coils 312 and/or the antennas 314. In some embodiments, the patch component 302 includes a mechanical housing 318 for holding the magnets 310 the coils 312, the antenna 314, and/or the wearable electronics components 316.

In some embodiments, the relay component 304 includes additional electronics for supporting functionalities of the patch component 302, and for enabling data and power transfer between the patch component 302 and the computing device 140. In some embodiments, the relay component 304 is configured to anchor the wearable device 130 on or near the human subject 102, such as on clothing worn by the human subject, or on a wheelchair or mobility device of the human subject, or on a patient bed of the human subject.

FIG. 3B is a block diagram showing an example data path (represented by closed triangle arrows), power path (represented by two-edged arrows), and control path (represented by open triangle arrows) of a wearable device 130, in accordance with some embodiments.

Data path-wise, in some embodiments, the wearable device 130 includes a neural data receiver module 332 for receiving neural signals in the form of timestamped data packets 243 from the implant device 110. In some embodiments, the neural data receiver module 332 generates packetized combined data 333 by further packetizing the timestamped data packets 243 along with data from the wearable device 130 and control commands. The neural data receiver module 332 transmits the packetized combined data 333 to a computing device communication module 334 that is configured to transmit the packetized combined data 333 to the computing device 140 via a connection 336 of the computing device.

The wearable device 130 is connected to an electrical power source. For example, the wearable device 130 can be connected to an electrical power outlet or can include an energy storage device and/or a battery that is configured to supply power to the wearable device 130. In some embodiments, the wearable device 130 includes a RF transmitter module 324 that is configured to transmit electrical power to the implant device 110 (e.g., to the RF power distribution module 322). Ad depicted in FIG. 3B, the electrical power source of the wearable device 130 is configured to provide power to various modules of the wearable electronics 316, including an initialization communication module 326, a system management module 328, a control layer communication module 330, the neural data receiver module 332, and the computing device communication module 334.

In some embodiments, the initialization communication module 326 is configured to set up and prepare the communication channels and protocols needed for the wearable device 130 to interact with the implant device 110 at the beginning of its operation.

In some embodiments, the system management module 328 is configured to manage and monitor the performance, health, and operation of the wearable device 130. For example, the system management module 328 can track system parameters such as temperature, voltage, and power consumption to ensure stable operation of the wearable device 130. In some instances, the system management module 328 can identify system failures, log errors, and provide alerts for troubleshooting.

In some embodiments, the control layer communication module 330 is configured to manage the flow of data between different modules of the wearable device 130 and between the wearable device 130 and the implant device 110.

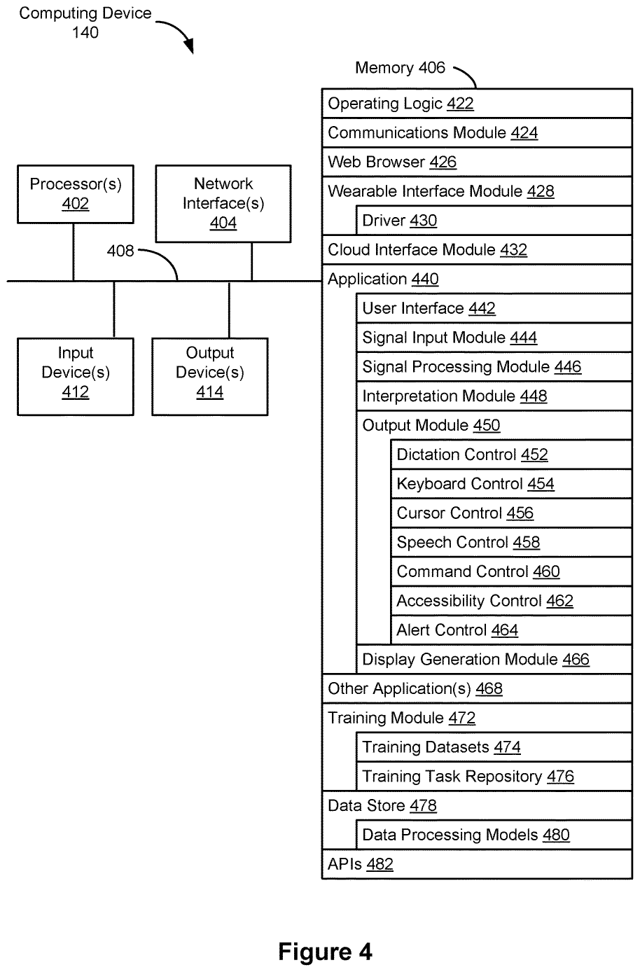

FIG. 4 is a block diagram illustrating a computing device 140, in accordance with some embodiments. Various examples of the computing device 140 include a desktop computer, a laptop computer, a tablet computer, a virtual reality (VR) device, an augmented reality (AR) device, or a spatial computing device that blends digital content with the physical world. The computing device 140 includes one or more processors 402 (e.g., processing units (CPUs) or cores), one or more network or communication interfaces 404, memory 406, and one or more communication buses 408 for interconnecting these components. In some embodiments, the communication buses 408 include circuitry (sometimes called a chipset) that interconnects and controls communications between system components. In some embodiments, the computing device 140 includes a user interface 410. The user interface 410 typically includes a display (e.g., display device) 412.

In some embodiments, the computing device 140 includes one or more input devices 412, which facilitate user input, such as a keyboard, a mouse, a voice-command input unit or microphone, a display 170 (e.g., which can be a touch-sensitive display), a touch-sensitive input pad, a gesture capturing camera, or other input buttons or controls. In some embodiments, the computing device 140 uses a microphone and voice recognition or a camera and gesture recognition to supplement or replace the keyboard. In some implementations, the computing device 140 includes one or more cameras, scanners, or photo sensor units for capturing images. In some embodiments, the computing device 140 includes one or more output devices 414, which enable presentation of user interfaces and display content, including one or more speakers and/or one or more visual displays.

The memory 406 includes high-speed random-access memory, such as DRAM, SRAM, DDR RAM, or other random-access solid-state memory devices. In some embodiments, the memory 20406 6 includes non-volatile memory, such as one or more magnetic disk storage devices, one or more optical disk storage devices, one or more flash memory devices, or one or more other non-volatile solid state storage devices. In some embodiments, the memory 406 includes one or more storage devices remotely located from the processors 402. The memory 406, or alternatively the non-volatile memory devices within the memory 406, includes a non-transitory computer-readable storage medium. In some embodiments, the memory 406, or the computer-readable storage medium of the memory 406, stores the following programs, modules, and data structures, or a subset or superset thereof:
an operating system 422, which includes procedures for handling various basic system services and for performing hardware dependent tasks;
a communications module 424, which connects the computing device 140 to other devices (e.g., various servers in the server system 160, implant device 110, wearable device 130, external display device 173, and mouse 174) via one or more network interfaces 404 (wired or wireless) and one or more networks 150, such as the Internet, other wide area networks, local area networks, metropolitan area networks, and so on;
a web browser 426 (or other application capable of displaying web pages), which enables a user to communicate over a network with remote computers or devices;
a wearable interface module 428, for interfacing with various modules and components of the wearable device 130. In some embodiments, the wearable interface module 428 includes:
   a driver 430, which provides two-way telemetry between the computing device and the wearable device 130 and data input for application 440;
a cloud interface module 432, for providing two-way communication between the computing device 140 and the server system 160;
an application 440. In some embodiments, the application 440 includes:
   a user interface 442 for providing visual information to users and for capturing user inputs;
   a signal input module 444 for receiving brain signals (e.g., electrical signals or data streams) of a human subject 102. As discussed with reference to FIGS. 1, 2, and 3, in some embodiments, the brain signals are collected by implant device 110 and transmitted to the wearable device 130. In some embodiments, the brain signals include attempted or intended speech signals (e.g., the human subject 102 tries to speak, thereby producing electrical signals corresponding to the intended movement of specific individual muscles or groups of muscles used for speech articulation, but their speech may not be vocalized due to underlying pathology preventing correct movement of the intended muscles or muscle groups). In some embodiments, the brain signals include attempted non-speech motor signals, such as attempted head, arm, hand, foot or leg movement signals (e.g., the human subject 102 tries to move their head or limbs, thereby producing electrical signals corresponding to the intended movement of specific individual muscles or groups of muscles used for the desired movement, but actual, physical movement may or may not occur due to underlying pathology preventing correct movement of the intended muscle or muscle groups) and attempted gestures (e.g. a hand squeeze or gesture);
   a signal processing module 446 for decoding the brain signals, including extracting electrophysiological features from the brain signals and performing signal processing functions. The extracted features represent brain activities intended for desired actions. In some embodiments, the signal processing module 446 analyzes signal characteristics such as amplitude, latency of event-evoked potentials, frequency power spectra (e.g., sensorimotor rhythms), or neuronal firing rates, in time-domain and/or frequency-domain. In some embodiments, the signal processing module 446 applies mathematical operations to enhance, filter, or modify the signals, allowing for better interpretation and utilization of the data;
   an interpretation module 448 (e.g., decoding module) for decoding the brain signals by recognizing patterns of the extracted features and the intended actions they represent. In some embodiments, the interpretation module 448 implements a classification process to recognize patterns in the extracted features. In some embodiments, the interpretation module 448 applies one or more machine learning models (e.g., data processing models 480 and/or data processing models 568) to decode the brain signals and determine intended actions of the human subject 102. In some embodiments, the interpretation module 448 applies a machine learning algorithm to decode the brain signals and determine intended actions of the human subject 102. In some embodiments, the machine learning algorithm is associated with the one or more machine learning models. In some embodiments, the interpretation module 448 is configured to generate a respective distinct set of intended actions depending on the operating mode (e.g., operating modes 190) of the computing device. In some embodiments, the interpretation module 448 is configured to translate and/or transform the classified features into actual commands;
   an output module 450 for controlling one or more devices, applications, and/or application interfaces to execute the one or more intended actions. In some embodiments, the output module 450 is configured to control the one or more devices, applications, and/or application interfaces to perform dictation control 452, keyboard control 454, cursor control 456, speech control 458, command control 460, accessibility control 462, and alert control 464. In some embodiments, the output module 450 is configured to control an external robotic arm. In some embodiments, the output module 450 is configured to control a prosthetic limb of the human subject 102; and
   a display generation module 466 for generating visual outputs for display on a display device such as a display screen 170 of the computing device 140 of an external display device 173;
one or more other applications 468 that are executed on the computing device 140, Exemplary applications can include a messaging application, an email application, a data presentation and communication application;
a training module 472 for performing limited machine learning model training. In some embodiments, the server system 160 is configured to train one or more machine learning models (e.g., decoding models) that are deployed locally on the computing device 140. In some embodiments, limited model training occurs locally on the computing device 140. In some embodiments, the training module 472 includes:
   one or more training datasets 474; and
   a training task repository 476, which includes training activities or tasks for training the models one or more data processing models 480.
a data store 478 for storing data associated with the computing device 140, including:
   one or more data processing models 480 (e.g., machine learning models, machine learning algorithms, decoding models, and/or decoding algorithms) for supporting the functions performed by the signal processing module 446 and/or the interpretation module 448. As used herein, a data processing model 480 can refer a machine learning model or an algorithm; and
APIs 482 for receiving API calls from one or more applications (e.g., a web browser 426, application 440, other applications 468), translating the API calls into appropriate actions, and performing one or more actions.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various embodiments. In some embodiments, the memory 406 stores a subset of the modules and data structures identified above. Furthermore, the memory 406 may store additional modules or data structures not described above. In some embodiments, a subset of the programs, modules, and/or data stored in the memory 406 is stored on and/or executed by a server system 160.

Although FIG. 4 shows a computing device 140, FIG. 4 is intended more as a functional description of the various features that may be present rather than as a structural schematic of the embodiments described herein. In practice, and as recognized by those of ordinary skill in the art, items shown separately could be combined and some items could be separated. In addition, some of the programs, functions, procedures, or data shown above with respect to the computing device 140 may be stored or executed on a server system 160.

FIG. 5 is a block diagram of a server system 160, in accordance with some embodiments. The server system 160 typically includes one or more processors 502 (e.g., processing units or cores (CPUs)), one or more network interfaces 504, memory 506, and one or more communication buses 508 for interconnecting these components (sometimes called a chipset). In some embodiments, the server system 160 includes one or more input devices 510, which facilitate user input, such as a keyboard, a mouse, a voice-command input unit or microphone, a touch screen display, a touch-sensitive input pad, a gesture capturing camera, or other input buttons or controls. In some embodiments, the server system 160 uses a microphone and voice recognition or a camera and gesture recognition to supplement or replace the keyboard. In some implementations, the server system 160 includes one or more cameras, scanners, or photo sensor units for capturing images. In some implementations, the server system 160 includes one or more output devices 512, which enable presentation of user interfaces and display content, including one or more speakers and/or one or more visual displays.

The memory 506 includes high-speed random access memory, such as DRAM, SRAM, DDR RAM, or other random access solid state memory devices. In some implementations, the memory 506 includes non-volatile memory, such as one or more magnetic disk storage devices, one or more optical disk storage devices, one or more flash memory devices, or one or more other non-volatile solid state storage devices. In some implementations, the memory 506 includes one or more storage devices remotely located from the processing units 502. The memory 506, or alternatively the non-volatile memory within the memory 506, includes a non-transitory computer readable storage medium. In some implementations, the memory 506, or the non-transitory computer readable storage medium of the memory 506, stores the following programs, modules, and data structures, or a subset or superset thereof:
an operating system 514, which includes procedures for handling various basic system services and for performing hardware dependent tasks;
a network communication module 516, which connects the server system 160 to other devices (e.g., various servers in the server system 160, computing device 140, a client device, or other devices) via one or more network interfaces 504 (wired or wireless) and one or more networks 150, such as the Internet, other wide area networks, local area networks, metropolitan area networks, and so on;
a user interface module 518, which enables presentation of information (e.g., a graphical user interface for presenting applications, widgets, websites and web pages thereof, and/or games, audio and/or video content) at a computing device 140 or electronic device 178;
a web browser module 520 for navigating, requesting (e.g., via HTTP), and displaying websites and web pages thereof, including a web interface for logging into a user account associated with a computing device 140 or another electronic device, controlling the computing device or electronic device if associated with the user account, and editing and reviewing settings and data that are associated with the user account;
an operational pipeline 524, including:
   a data intake module 526 for obtaining respective neural signals that are collected by respective implant devices;
   a data selection module 528 for automatically choosing the most relevant and suitable data subsets from a larger data pool (e.g., form the one or more databases 162);
   a data processing module 538 for transforming raw sensor data 576 through various processing steps such as data cleaning, normalization, and feature extraction. In some embodiments, the data processing module 538 generates training data for use by a model training module 532 by preparing the processed data in a suitable format;
   the model training module 532 for receiving the training data and establishing one or more data processing models 568 for processing neural data collected by implant devices 110. In some embodiments, the model training module 532 includes labels 534 for training the data processing models 568. In some embodiments, the model training module 532 includes training datasets 536 for training the data processing models 568;
a web application 540, which may be downloaded and executed by a web browser 426 on a user's computing device 140. In general, a web application 540 has the same functionality as application 440, but provides the flexibility of access from any device at any location with network connectivity, and does not require installation and maintenance. In some embodiments, the web application 540 includes various software modules to perform certain tasks, such as:
   a user interface 542, which provides the user interface for all aspects of the web application 540;
   a signal input module 544, which has the same functionalities as signal input module 444
   a signal processing module 546, which has the same functionalities as signal processing module 446;
   an interpretation module 548, which has the same functionalities as interpretation module 448;
   an output module 550, which has the same functionalities as output module 450; and
   a display generation module 552, which has the same functionalities as display generation module 466;
one or more databases 162 for storing at least data including one or more of:
   device settings 562 including common device settings (e.g., service tier, device model, storage capacity, processing capabilities, communication capabilities, etc.) of the one or more server systems 160, computing devices 140, or other devices;
   user account information 564 for the application 440 and/or the web application 540, such as user names, security questions, account history data, user preferences, and predefined account settings
   application data 566 from the application 540, web application 540, and/or the user applications
   firmware data 567 corresponding to respective firmware versions;
   data processing models 568 (e.g., machine learning models, machine learning algorithms, decoding models, and/or decoding algorithms) for decoding neural signal data into intended user actions. In some embodiments, the data processing models 568 include model parameters 570, weights 572, and metadata 574
   raw sensor data 576 acquired by one or more electrode arrays 208;
   derived data 578 obtained by transforming, processing, or analyzing the raw sensor data 576; and
   analytics data 580, which are data that are collected, processed, and analyzed from the raw sensor data 576 and the derived data 578 to gain insights and identify patterns; and
APIs 590 for receiving API calls from one or more applications (e.g., web browser module 520, user applications 522, and web application 540) and the one or more data processing models 568, translating the API calls into appropriate actions, and performing one or more actions.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various embodiments. In some embodiments, the memory 506 stores a subset of the modules and data structures identified above. Furthermore, the memory 506 may store additional modules or data structures not described above.

Although FIG. 5 shows a server system 160, FIG. 5 is intended more as a functional description of the various features that may be present rather than as a structural schematic of the embodiments described herein. In practice, and as recognized by those of ordinary skill in the art, items shown separately could be combined and some items could be separated. In addition, some of the programs, functions, procedures, or data shown above with respect to a server system 160 may be stored or executed on a computing device 140. In some embodiments, the functionality and/or data may be allocated between a computing device 140 and one or more server systems 160. Furthermore, one of skill in the art recognizes that FIG. 5 need not represent a single physical device. In some embodiments, the server functionality is allocated across multiple physical devices in a server system. As used herein, references to a “server” include various groups, collections, or arrays of servers that provide the described functionality, and the physical servers need not be physically colocated (e.g., the individual physical devices could be spread throughout the United States or throughout the world).

### BCI Systems With Magnetic Arrangement for Alignment

As described above, the implant device 110 is configured to couple to the wearable device 130 (e.g., patch component 302 and/or relay component 304), and transmit data from the channels of the electrode array 208 wirelessly to the wearable device 130. In accordance with some embodiments, to achieve faster (e.g., real-time or near real time), more accurate, and more natural communication, the BCI system includes a high-density electrode array with high electrode channel count (e.g., at least 1000 electrode channel counts). Accordingly, the implant device 110 has to transmit at least 1000 electrode channels of neural signal data in real time to the wearable device 130. Accordingly, there is a need for improved methods and devices that can enable precise alignment between the implant device 110 (e.g., one or more antennas of the implant device) and the wearable device 130, to facilitate efficient wireless data communication between the two devices at a higher data rate connection.

In accordance with some embodiments, the implant device 110 does not have a battery. In some embodiments, the implant device 110 is configured to receive power wirelessly from the wearable device 130 (e.g., via inductive coupling or capacitive coupling). Accordingly, there is also a need for improved methods and devices that enable precise alignment between the implant device 110 and the wearable device 130, to enable higher fidelity power connection.

Some embodiments of the present disclosure provide a technical solution to the aforementioned technical problems by implementing an implant device that includes a frame that is configured to be anchored to a skull of a patient. The frame includes one or more channels (e.g., slots) positioned along at least a part of the perimeter of the frame (e.g., on one or more sides of the frame). The channels are configured to hold one or more magnets within the channels. In some embodiments, the one or more magnets can be securely attached within one or more hermetically sealed packages (e.g., by welding, adhesives, or other attachment means), which are then placed inside the one or more channels.

In some embodiments, the one or more channels are positioned at at least two sides of the frame and the magnets are positioned within the at least two sides of the frame.

The magnets positioned within the at least two sides of the frame magnetically couple to complementary magnets that are positioned at (e.g., in) the patch device 302, thus enabling coupling between the implant device 110 and the wearable device 130. The number, and strength, of the magnets provide the retention force (e.g., magnetic attraction force) needed to keep the patch component 302 in place

In some embodiments, when arranged on two or more sides of the frame, the magnets of the implant device provide alignment in multiple axes, including the x-axis, y-axis, and rotation, pitch, and roll with respect to an xy-plane.

In some embodiments, the one or more channels of the frame can be positioned on two adjacent sides of the frame to form an L-shaped channel that is configured to hold magnets in the L-shape arrangement. In some embodiments, the one or more channels can be positioned on two opposite sides of the frame. In some embodiments, the one or more channels are positioned on three adjacent sides of the frame to form a U-shape arrangement of magnets. In some embodiments, the one or more channels are positioned on all sides of the frame to form a ring-or annular-shaped arrangement of magnets.

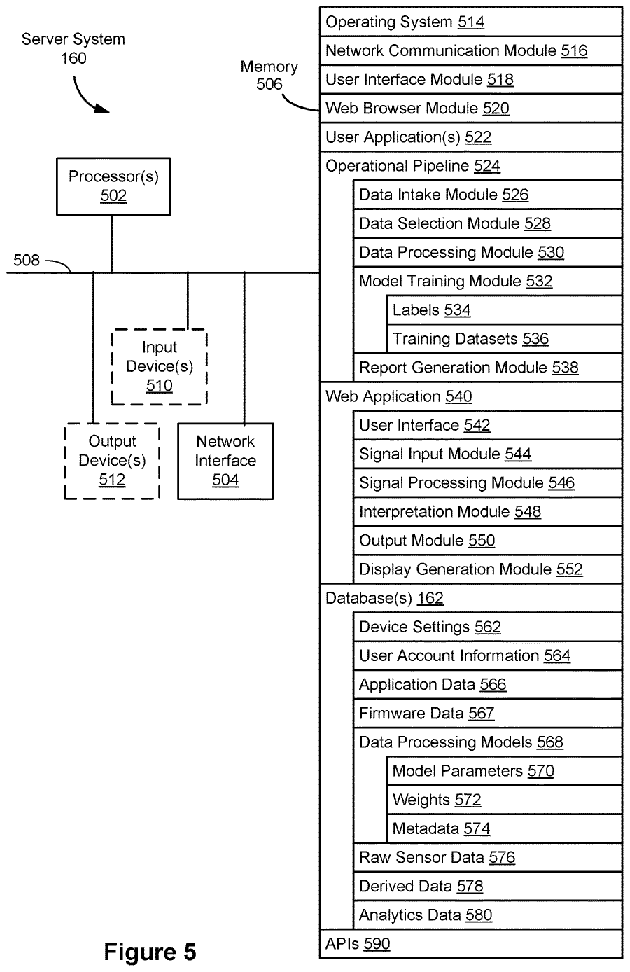

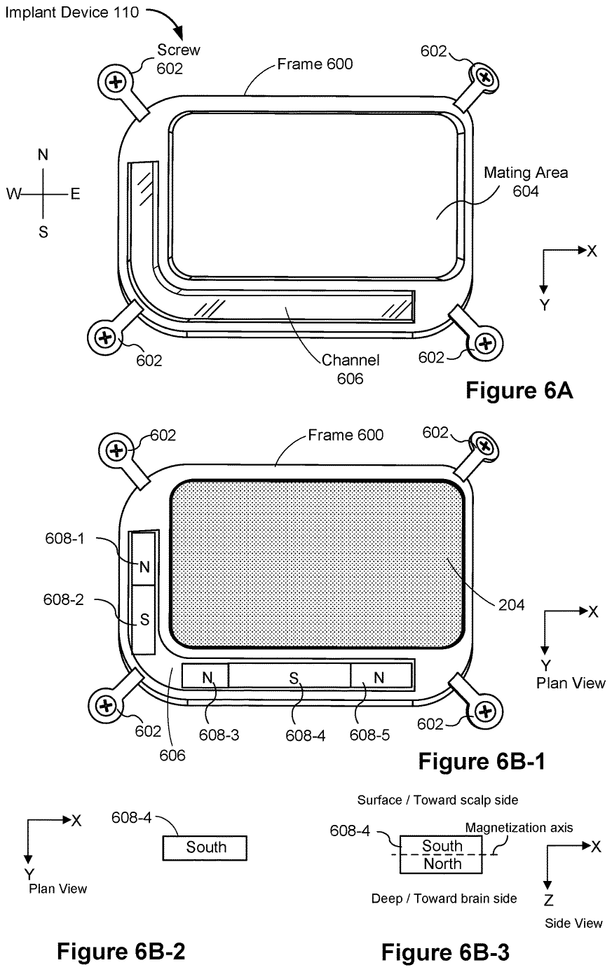

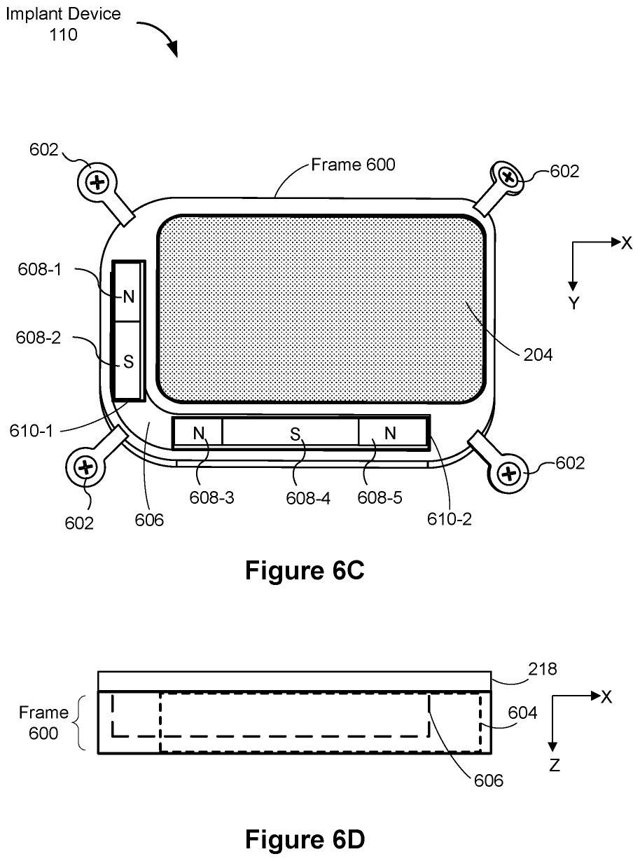

FIGS. 6A, 6B-1, 6B-2, 6B-3, 6C, and 6D are various views illustrating an implant device 110 (e.g., a cranial implant device), in accordance with some embodiments. The implant device 110 includes a frame 600. The implant device 110 includes a plurality of screws 602. In the example of FIG. 6A, the screws 602 are positioned at the four corners of the implant device 110. The screws 602 are configured to anchor the frame 600 to a skull of a patient (e.g., human subject 102).

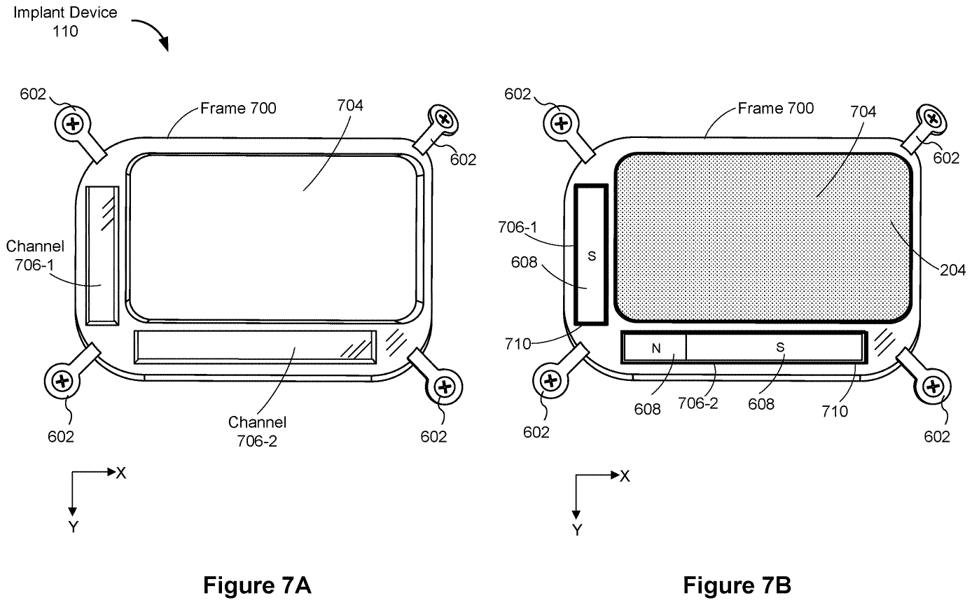

In some embodiments, the frame 600 includes a mating area 604 (e.g., a recessed area). In some embodiments, the dimensions (e.g., length width, and/or height) of the mating area are configured so as to enable the hermetic package 204 to mate with (e.g., fit within) the mating area 604, as illustrated in FIGS. 6B-1 and 6C. In the example of FIG. 6 (and also in FIGS. 7-12), the hermetic package 204 is removable from the frame. In other embodiments, the hermetic package 204 may be fixedly coupled to the frame (i.e., not removable).

In some embodiments, the frame 600, the screws 602, and/or the hermetic package 204 are made of biocompatible materials such as stainless steel, titanium and titanium alloys, silicone, ceramic or polymer materials or a combination thereof.

In some embodiments, the frame 600 includes a channel 606. In the example of FIGS. 6A to 6D, the channel 606 comprises an L-shaped channel that is positioned along two adjacent (e.g., orthogonal) sides of the frame.

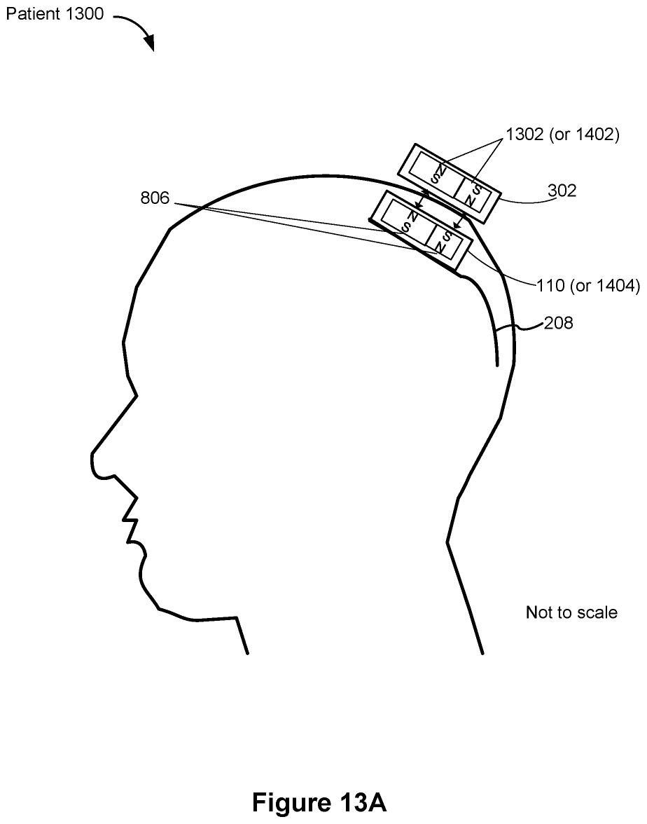

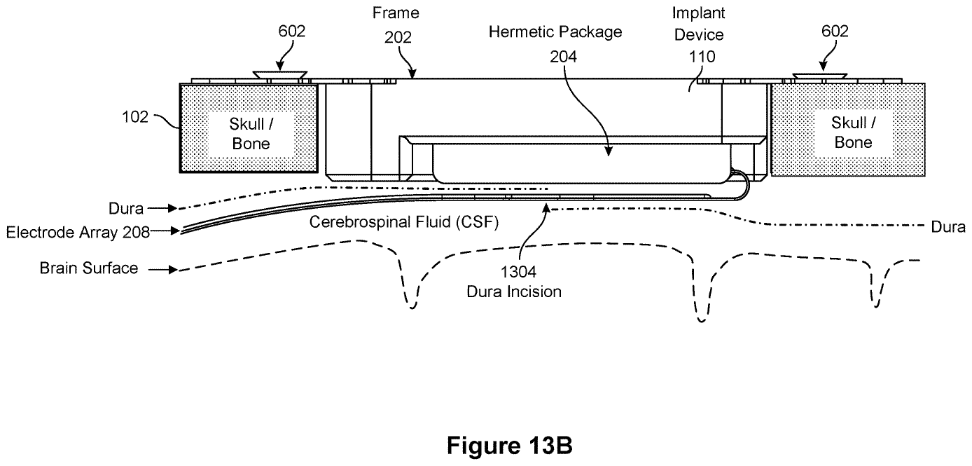

In some embodiments, the channel 606 is configured to hold one or more magnets 608. In some embodiments, the magnets 608 are configured to magnetically couple to one or more complementary magnets 1302 that are positioned at the patch device 302, as illustrated in FIG. 13A.

The example of FIG. 6B-1 shows a plan view of the implant device 110 where two magnets 608-1 and 608-2 are positioned at the shorter side of the channel 606 and three magnets 608-3 to 608-5 are positioned at the longer side of the channel 606. Adjacent magnets of the plurality of magnets 608 have opposite (e.g., reverse) polarities. In some embodiments, the shorter and longer sides of the channel 606 are orthogonal to each other and causes the magnets 608 positioned at both sides to be orthogonal, thus fixing the alignment in two dimensions. In some embodiments, instead of using multiple magnets, a stronger magnet with a volume that is similar to the combined volume of the two magnets can be placed at the shorter side of the channel, and another stronger magnet with a volume that is similar to the combined volume of the three magnets can be placed at the longer side of the channel. FIGS. 6B-2 and 6B-3 show, respectively, a plan view and side view of the magnet 608-4. FIG. 6B-3 shows the axis of magnetization of the magnet 608-4.

In accordance with some embodiments, the tradeoff between having a fewer number of larger-sized magnets and a bigger number of smaller-sized magnets is that a larger-sized magnet has a stronger magnetic attractive force and couples more strongly to the patch component 302, but multiple smaller magnets, especially when positioned in two or more sides, enable better alignment between the implant device 110 and the patch component 302. In some embodiments, the magnets 608 have the same size. In some embodiments, at least one magnet has a different size from the rest of the magnets. In some embodiments, all the magnets 608 have different sizes.

In some embodiments, the magnets 608 are welded into one or more hermetically sealed packages that are configured to fit within the channel 606. FIG. 6C illustrates an example where the two magnets 608-1 and 608-2 on the shorter side of the channel 606 are welded within a hermetically sealed package 610-1 and the three magnets 608-3 to 608-5 on the longer side of the channel 606 are welded within a hermetically sealed package 610-2. The hermetically sealed package 610-1 and the hermetically sealed package 610-2 are configured to fit within the dimensions of the channel 606. In some embodiments, all the magnets 608 that are configured to be positioned within the channel 606 can be welded within one hermetically sealed package. In some embodiments, the hermetically sealed package 610-1 and the hermetically sealed package 610-2 are made of a biocompatible material such as titanium or silicone. FIG. 6D shows a side view of the frame 600 (e.g., in the x-z plane) with a cover 218 mounted on the top of the frame 600, in accordance with some embodiments.

FIGS. 7, 8, 9, 10, 11, and 12 illustrate different frame and channel configurations of the implant device 110, in accordance with some embodiments.

In the example of FIGS. 7A and 7B, the implant device 110 includes a frame 700. The frame 700 includes a mating area 704 that is configured to mate with the hermetic package 204. Details of the mating area are as discussed above with respect to FIG. 6, and not repeated for the sake of brevity. The frame 700 includes a channel 706-1 and a channel 706-2 (i.e., two channels) that are positioned at two adjacent sides of the frame 700. The channels 706-1 and 706-2 are configured to hold magnets 608. The magnets 608 may be packaged in one or more hermetically sealed packages 710 that are configured to fit within the channel 706.

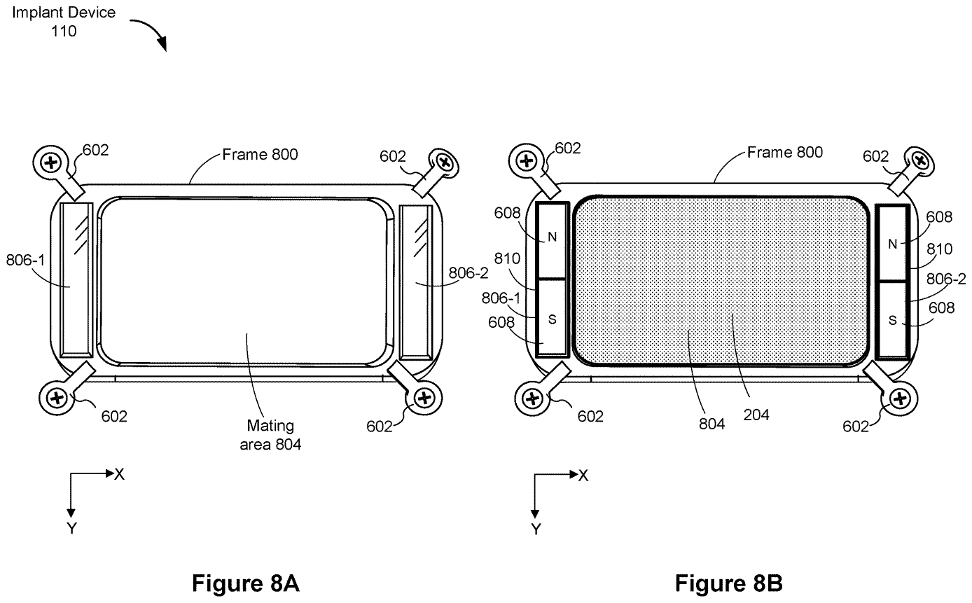

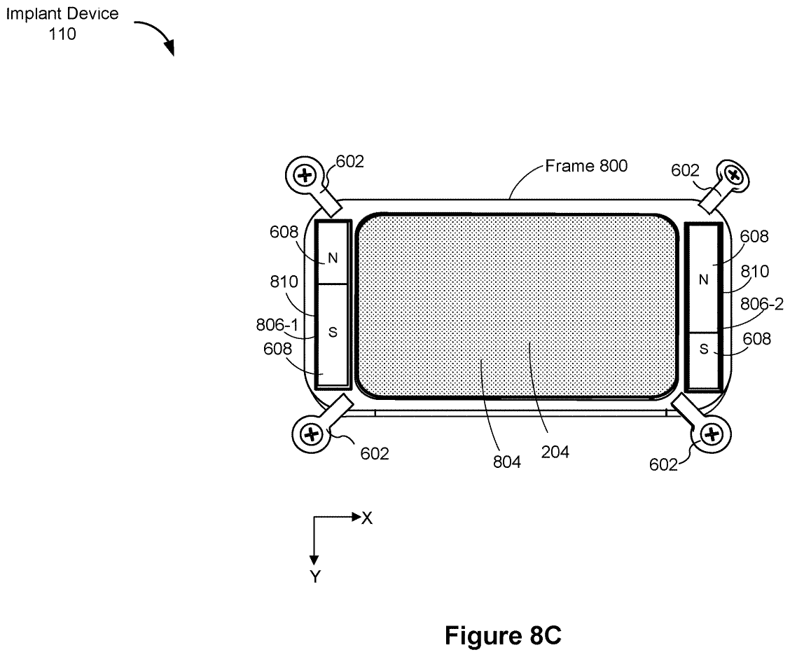

In the example of FIG. 8A 8B, and 8C, the implant device 110 includes a frame 800. The frame 800 includes a mating area 804 that is configured to mate with the hermetic package 204. The frame 800 includes a channel 806-1 and a channel 806-2 (i.e., two channels) that are positioned on opposite sides of the frame 800. The channels 806-1 and 806-2 are configured to hold magnets 608. In some embodiments, the magnets 608 are enclosed in hermetically sealed packages 810. In some embodiments, the magnets 608 have the same dimensions (i.e., are equal-sized), as shown in the top-down view in FIG. 8B. In some embodiments, the magnets 608 are not of equal dimensions, as shown in the top-down view in FIG. 8C.

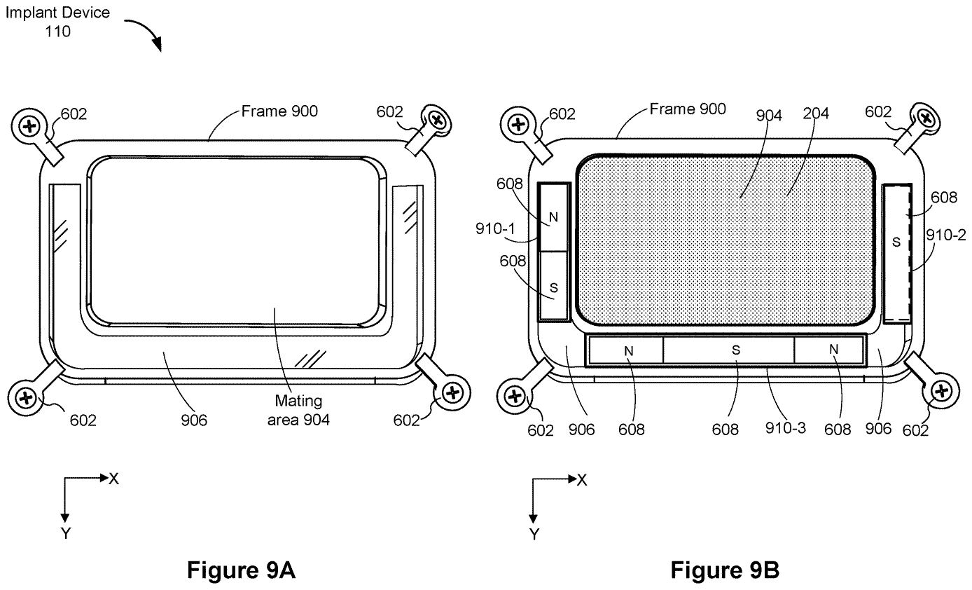

FIG. 9A shows an implant device 110, in accordance with some embodiments. In the example of FIG. 9A, the implant device includes a frame 900 with a mating area 904 and a U-shaped channel 906 that spans three adjacent sides of the frame 900. FIG. 9B illustrates the hermetic package 204 positioned within the mating area 904. Magnets 608 are positioned within the U-shaped channel 906. In some embodiments, the magnets 608 are enclosed in one or more hermetic packages 910 (e.g., hermetic package 910-1 to 910-3).

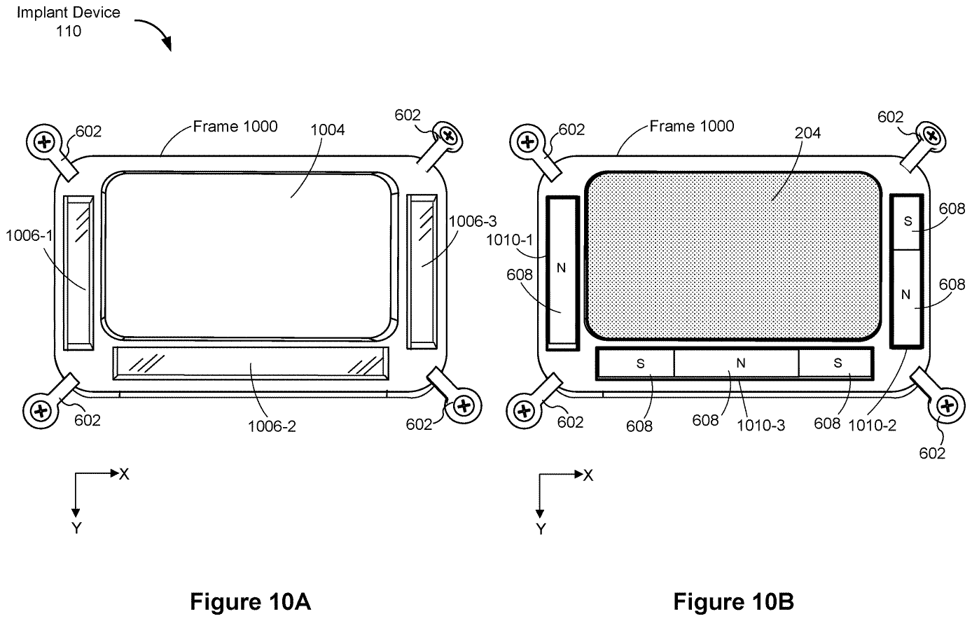

In the example of FIGS. 10A and 10B, the implant device 110 includes a frame 1000. The frame 1000 includes a mating area 1004 that is configured to mate with the hermetic package 204. The frame 1000 includes three distinct channels 1006-1, 1006-2, and 1006-3, which are positioned at three adjacent sides of the frame 1000. The channels 1006-1, 1006-2, and 1006-3 are configured to hold magnets 608. In some embodiments, the magnets 608 are enclosed in hermetically sealed packages 1010 (e.g., hermetically sealed package 1010-1 to 1010-3).

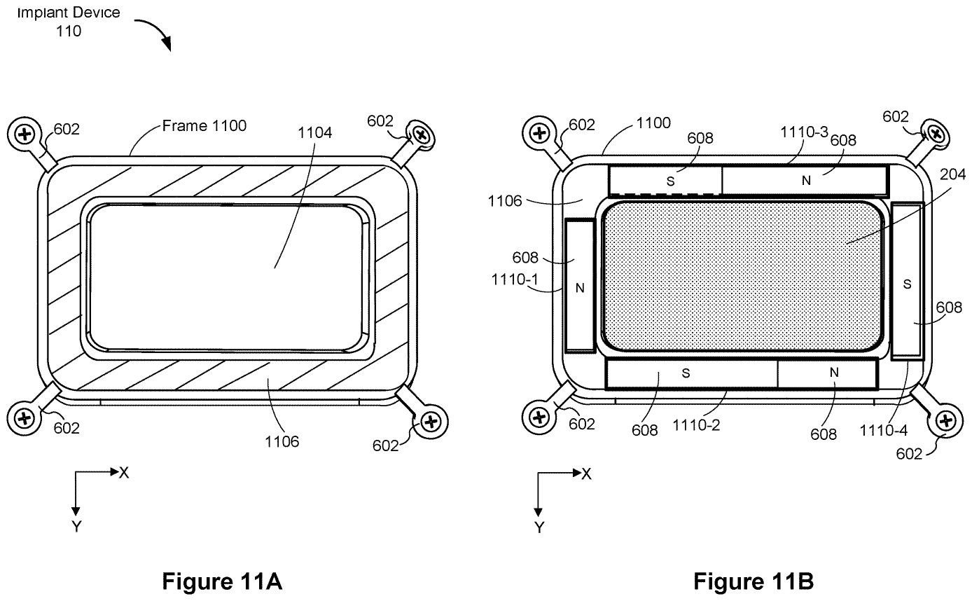

In the example of FIGS. 11A and 11B, the implant device 110 includes a frame 1100. The frame 1100 includes a mating area 1104 that is configured to mate with the hermetic package 204. The frame 1100 includes one channel 1106 that surrounds all sides of the frame 1100 and is configured to hold magnets 608. FIG. 11B illustrates magnets positioned along the four sides of the channel 1106. In some embodiments, the magnets 608 are enclosed in hermetically sealed packages 1110 (e.g., hermetically sealed package 1110-1 to 1110-4).

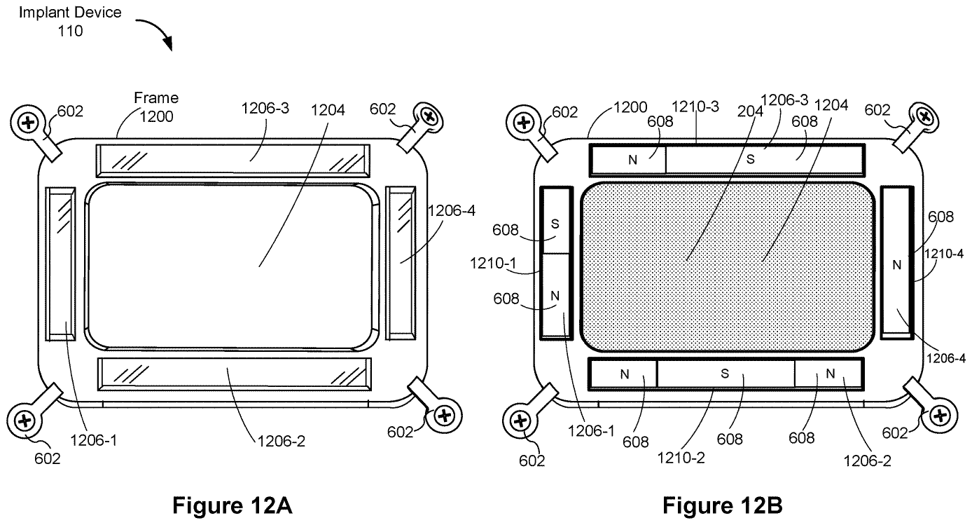

In the example of FIGS. 12A and 12B, the implant device 110 includes a frame 1200. The frame 1200 includes a mating area 1204 that is configured to mate with the hermetic package 204. The frame 1200 includes four channels 1206-1 to 1206-4, where each channel is positioned along a respective side of the frame 120. A respective channel 1206 is configured to hold one or more magnets 608. In some embodiments, the magnets 608 are enclosed in hermetically sealed packages 1210 (e.g., hermetically sealed package 1210-1 to 1210-4).

In some embodiments, the hermetic package 204 has a predefined size. Consequently, in some embodiments, the mating areas 604, 704, 804, 904, 1004, 1104, and 1204 depicted in FIGS. 6 to 12 have the same area (e.g., a fixed area or volume).

In some embodiments, an area of the frame varies according to the configuration of the channels. For example, the dimensions (e.g., length and width), area, and/or volume of the frame (e.g., frame 600, 700, 800, 900, 1000, 1100, or 1200) can vary depending on the dimensions and/or configuration of the channels. In some embodiments, a length of the frame is around 40 mm, 41 mm, 42 mm, 43 mm, 44 mm, 45 mm, 46 mm, or 47 mm. In some embodiments, a width of the frame is about 30 mm, 31 mm, 32 mm, 33 mm, 34 mm, 35 mm, or 36 mm.

In some embodiments, a depth of the channel (e.g., channel 606, 706, 806, 906, 1016, 1106, or 1206) is around 3 mm, 4 mm, 5 mm, 6 mm, 7 mm, 8 mm, 9 mm, 10 mm, 11 mm, 12 mm, 13 mm, 14 mm, or 15 mm.

FIG. 13A illustrates a side view of a patient 1300 (e.g., human subject 102), in accordance with some embodiments. In FIG. 13, the implant device 110 is anchored to the skull of the patient 1300. The implant device 110 includes magnets 608 that are configured to magnetically couple to complementary magnets 1302 that are located at the patch component 302. The two-way arrows indicate magnetic coupling between the implant device 110 and the patch component 302.

In some embodiments, the frame (e.g., frame 600, 700, 800, 900, 1000, frame 1100, or 1200) is configured to be positioned toward a side of the skull, at a relatively flat region of the skull. In some embodiments, the electrode array is configured to extend from the frame toward the back of the skull, where the sensory motor cortex region is located. In some embodiments, the area of the frame is optimized such that its total footprint (e.g., area) is limited to a predefined region of the skull (e.g., a region toward the back of the skull) and the magnetic strength from the magnets 608 achieves the necessary alignment between the implant device (e.g., hermetic package) and the patch component 302.

FIG. 13B is a cross-sectional view depicting insertion of the implant device 110 into the skull of a human subject 102, in accordance with some embodiments. In some embodiments, the implant device 110, including at least a portion of the electrode array 208, is configured to be inserted into the skull of the human subject 102 via a craniectomy process. In the example of FIG. 13B, the frame 202 and the hermetic package 204 with the implant electronics are positioned above the dura, closer to the scalp of the human subject 102, whereas a portion of the electrode array 208 is inserted into the sub-dural layer (i.e., underneath the dura) of the brain of the human subject 102 The process includes making an dura incision 1304 on the dura layer and inserting a portion of the electrode array through the dura incision 1304, into the dura mater layer.

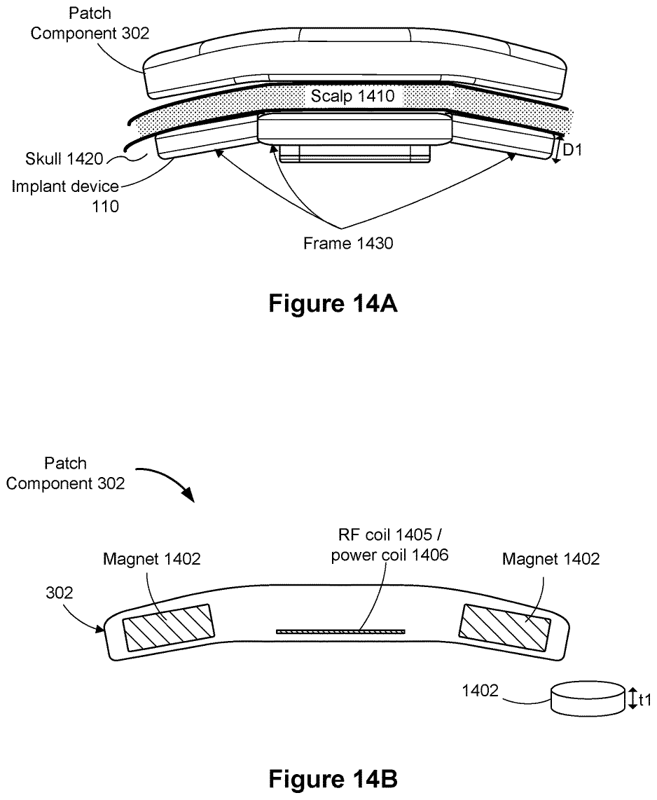

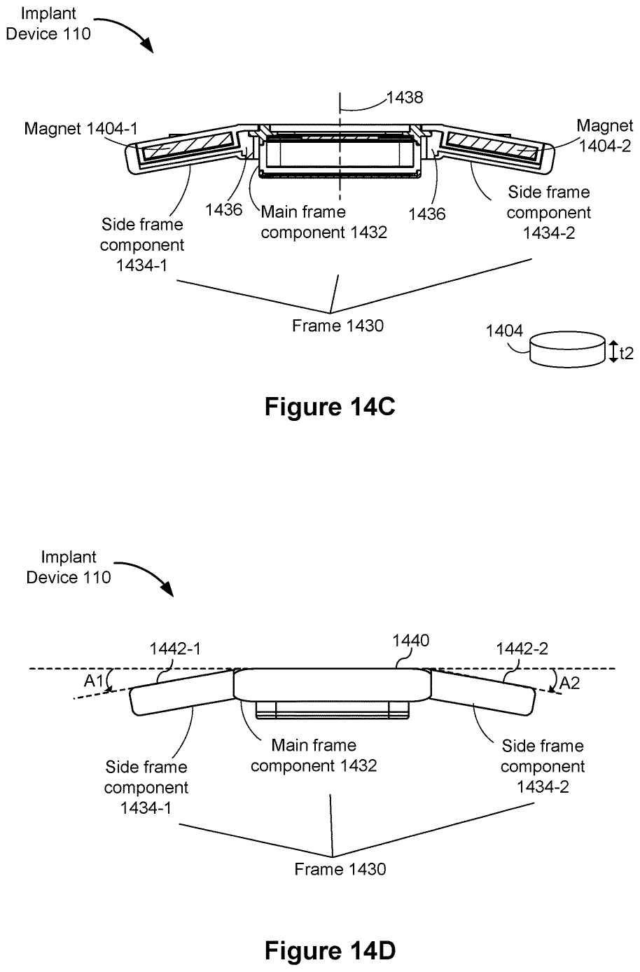

FIGS. 14A, 14B, and 14C illustrate a patch component 302 and an implant device 110, in accordance with some embodiments. FIG. 14A depicts the placement of the patch component 302 and the implant device 110 relative to a scalp 1410 and a skull 1420 of a human subject, in accordance with some embodiments. The patch component 302 is configured to be positioned above (e.g., outside) the scalp 1410 and at least a portion of the implant device 110 is configured to be positioned under skull 1420.

In some embodiments, and as illustrated in FIG. 14B, the patch component 302 (or a housing of the patch component) may have a contour that roughly conforms to the curvature of the scalp 1410 of the human subject 102. The patch component houses an RF coil 1405 (e.g., a RF antenna) for data transfer (e.g., for transferring neural data from the implant device 110 to the patch component 302). The patch component houses a power coil 1406 for power transfer (e.g., for transferring power from the patch component 302 to the implant device 110). In some embodiments, the power coil 1406 is an inductive coil. In some embodiments the RF coil 1405 and the power coil 1406 are the same coil. In some embodiments, the patch component 302 includes one or more magnets 1402 in FIG. 14B. In some embodiments, a thickness t1 (e.g., height) of the magnet 1402 does not exceed 1.5 millimeters (mm), 2.0 mm, 2.5 mm, 3.0 mm, 3.5 mm, 4.0 mm, 4.5 mm, or 5.0 mm. In some embodiments, a shape, size, thickness (t1), and/or magnetic strength of the magnet 1402 can be varied in order to tune the retention force between the implant device 110 and patch component 302. In some embodiments, the magnet 1402 has a cylindrical or disc shape with a diameter that is about 7 mm, 8 mm, 9 mm, 10 mm, 11 mm, 12 mm, 13 mm, 14 mm, 15 mm, 16 mm, or 17 mm. In the case where the magnet 1402 is a disc shaped magnet, thicker (e.g., larger t1) or larger-diameter magnets will have a stronger magnetic force, whereas thinner (e.g., smaller t1) or smaller-diameter magnets tend to have weaker magnetic force. Accordingly, different size magnets can be used to optimize a magnetic retention force between patch component 302 and implant device 110.

FIG. 14C shows the implant device 110 includes a frame 1430 that is configured to affix the implant device 110 to the skull. The frame 1430 may have a contour that is adapted to conform to the curvature of the brain of the human subject 102. In some embodiments, the frame 1430 includes a main frame component 1432 and one or more side frame components, such as the two side frame components 1434-1 and 1434-2 shown in FIG. 14B. Side frame component 1434 can also be referred to as a winged side, a side fin, or a lateral extension. In some embodiments, a side frame component 1434 is configured to house one or more magnets 1404. For example, FIG. 14B shows side frame component 1434-1 is configured to house magnet 1404-1 and side frame component 1434-2 is configured to house magnet 1404-2. In some embodiments, a respective magnet 1404 has a disc shape. In some embodiments, the main frame component 1432 and the side frame components 1434 can be made of a biocompatible metal such as titanium, titanium alloy, tantalum, nitinol (nickel-titanium), or stainless steel, a biocompatible plastic such as polyether ether ketone (PEEK), polyethylene (PE), polypropylene (PP), polycarbonate (PC), silicone, and polylactic acid (PLA), or any combination thereof.

In some embodiments, the magnets 1404 that are positioned in the implant device 110 are configured to magnetically couple to magnets 1402 that are positioned at the patch device 302, as illustrated in FIG. 13A.

In some embodiments, a respective magnet 1404 has a thickness t2 (e.g., height) that does not exceed 1.0 millimeter (mm), 1.1 mm, 1.2 mm, 1.3 mm, 1.4 mm, 1.5 mm, 1.6 mm, 1.7 mm, 1.8 mm, or 1.9 mm. In some embodiments, the magnet 1404 is a disc-shaped magnet with a thickness t2 of 1.4 mm. In some embodiments, the magnet 1404 is a disc-shaped magnet with a thickness t2 of 1.5 mm. In some embodiments, the magnet 1404 is a disc-shaped magnet with a thickness t2 of 1.6 mm. In some embodiments, the magnet 1404 is a disc-shaped magnet with a diameter of about 7 mm, 8 mm, 9 mm, 10 mm, 11 mm, 12 mm, 13 mm, 14 mm, 15 mm, 16 mm, or 17 mm. In some embodiments, the magnet 1404 has a predetermined (e.g., fixed size) or magnetic strength. In some embodiments, a size and/or magnetic strength of the magnet 1404 can be varied to optimize a magnetic retention force between patch component 302 and implant device 110.

Advantageously, in some embodiments, the magnets 1404 are sufficiently thin such that a shallow pocket in the skull (e.g., shallow pocket craniectomy), rather than a full craniectomy, may be created in the area of the side frame components 1434-1 and 1434-2 in order to implant the frame 1430 flush with the outer skull surface. The shallow pocket craniectomy creates a shallow recess or cavity in the skull bone without going all the way through the skull. FIG. 14A shows that the recess or cavity that is created in the skull has cavity depth of roughly D1, where D1 is up to about 4 mm, in accordance with some embodiments. In some instances, D1 may be shallower (e.g., less than 4 mm) to accommodate thinner bone. This scenario would cause the implant device 110 to be positioned slightly proud of skull surface (e.g., up to 2 mm), which is acceptable for surgical implants. By contrast, the depth of a full craniectomy is the thickness of the skull bone to allow a surgeon to slide at least part of the electrode array under the dura of the patient.

In some embodiments, the main frame component 1432 can be any one of frame 600, frame 700, frame 800, frame 900, frame 1000, frame 1100, or frame 1200. In some embodiments, the main frame component 1432 can include one or more channels that are configured to hold one or more magnets, such as the channels and magnets as shown and described with reference to FIGS. 6A to 6D, 7A, 7B, 8A to 8C, 9A, 9B, 10A, 10B, 11A, 11B, 12A, and 12B. These details are not repeated here for the sake of brevity.

In some embodiments, the frame 1430 is a unitary (e.g., monolithic or single piece) frame in which the one or more side frame components 1434 are integrally formed with the main frame component 1432. In some embodiments, the two magnets 1404-1 and 1404-2 are symmetrical about a centerline 1438 of the frame 202 (e.g., implant device 110). In some embodiments, the side frame components 1434-1 and 1434-2 are also symmetrical about the centerline 1438 of the frame 202.

In some embodiments, the one or more side frame components 1434 are discrete components that are movably and/or detachably coupled to the main frame component 1432 via one or more connecting components 1436. For example, the one or more side frame components 1434 may be coupled to the main frame component 1432 using one or more fasteners, interlocking features, adhesives, or other suitable attachment mechanisms. In this scenario, it may be possible to adjust a position of the side frame component 1434, relative to the main frame component, such that the side frame components 1434-1 and 1434-2 may not be symmetrical about the centerline 1438.

Referring now to FIG. 14D, an included angle between the top surface 1440 of the main frame component 1432 and the top surface 1442-1 of the side frame component 1434-1 is denoted as A1; and an included angle between the top surface 1440 of the main frame component 1432 and the top surface 1442-2 of the side frame component 1434-2 is denoted as A2. In some embodiments, the angle A1 is about 5°, 6°, 7°, 8°, 9°, 10°, 11°, 12°, 13°, 14°, or 15°. In some embodiments, the angle A2 is about 5°, 6°, 7°, 8°, 9°, 10°, 11°, 12°, 13°, 14°, or 15°. In some embodiments where the side frame components 1434-1 and 1434-2 are integrally formed with the main frame component 1432, the angles A1 and A2 are predetermined (i.e., fixed) during manufacturing and are not adjustable. In some embodiments where the side frame components 1434-1 and 1434-2 are movably coupled with the main frame component 1432, each of the angles A1 and A2 is independently adjustable (e.g., during the shallow pocket craniectomy process).

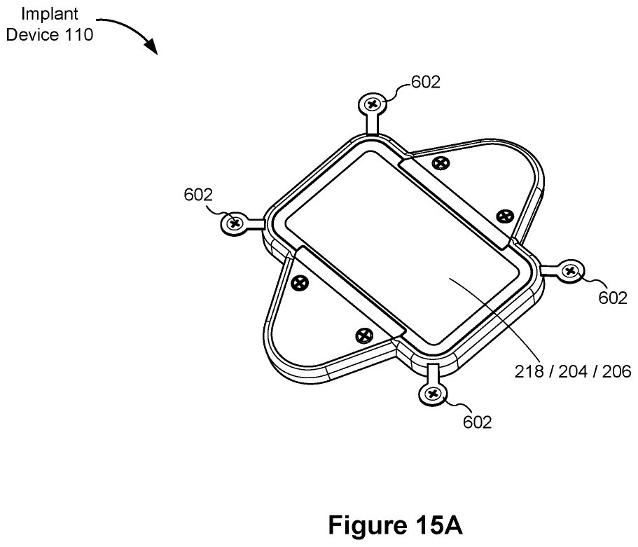

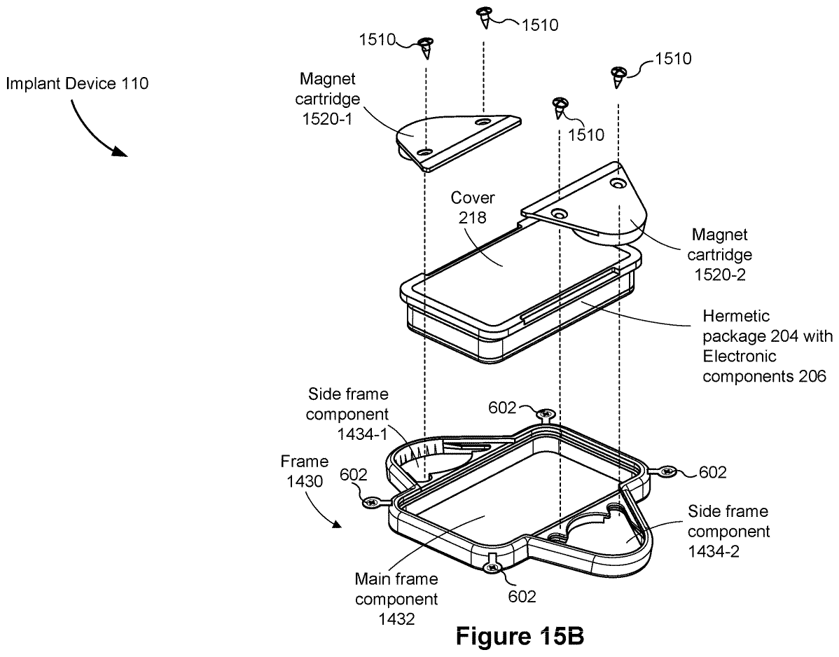

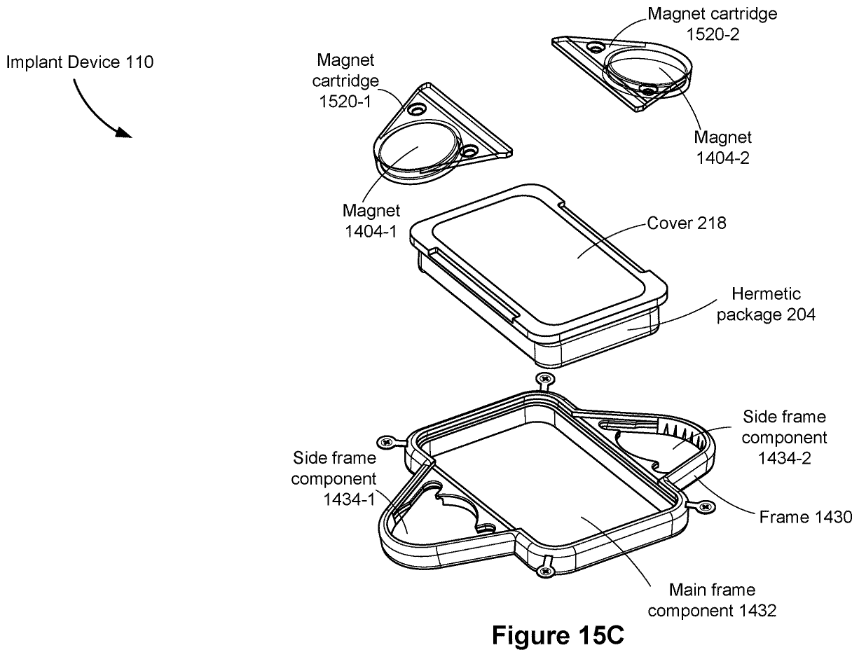

FIGS. 15A, 15B, and 15C illustrate perspective and exploded views of the implant device 110, in accordance with some embodiments. The implant device 110 includes a magnet cartridge 1520-1 that is configured to hold magnet 1404-1 and a magnet cartridge 1520-2 that is configured to hold magnet 1404-2. Magnet 1404 is configured to be encapsulated (e.g., sealed) inside the magnet cartridge 1520. Magnet cartridge 1520 creates a biocompatible barrier between the patient's body and the magnet 1404. A respective magnet cartridge 1520 can be made of a nonmagnetic, biocompatible metal such as titanium, titanium alloy, tantalum, nitinol (nickel-titanium), or stainless steel, or any combination thereof. Magnet cartridge 1520 (with the encapsulated magnet 1404) is configured to be removable from the patient. For example, before a magnetic resonance imaging (MRI) scan, the patient can undergo a secondary surgical procedure whereby the magnet cartridge 1520 with the encapsulated magnet 1404 are replaced with an empty cartridge, to prevent possible patient discomfort or device dislocation during MRI.

The main frame component 1432 includes a mating area (e.g., a recessed area) that is adapted to mate with hermetic package 204. The side frame component 1434-1 includes a mating area that is adapted to accommodate (e.g., house) the portion of the magnet cartridge 1520-1 holding magnet 1404-1. The side frame component 1434-2 includes a mating area that is adapted to accommodate the portion of the magnet cartridge 1520-2 holding magnet 1404-2. In some embodiments, one or more fasteners (e.g., screws) 1510 secure the magnet cartridge 1520-1 to the side frame component 1434-1 and secure the magnet cartridge 1520-2 to the side frame component 1434-2.

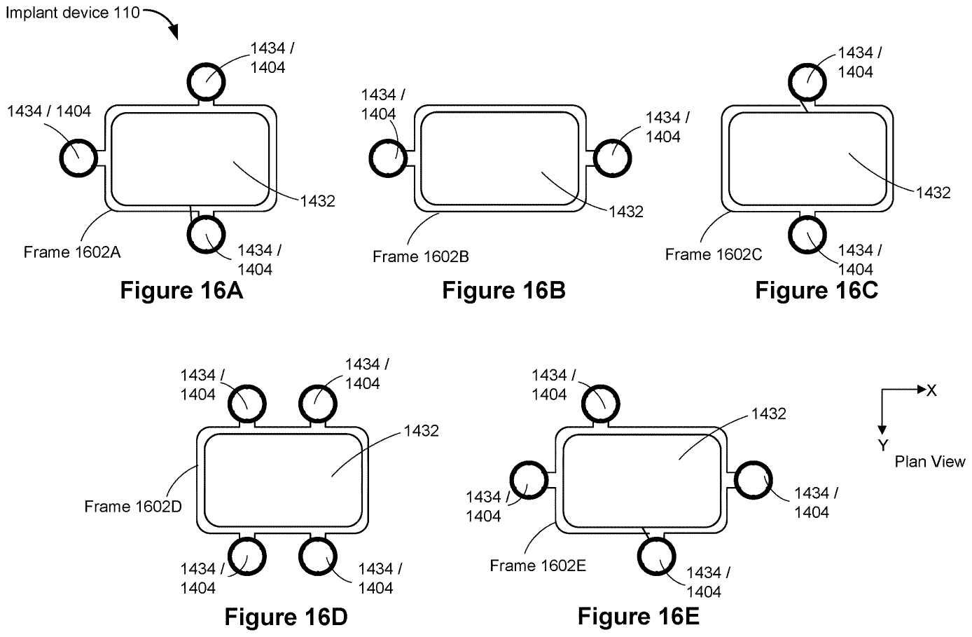

FIGS. 16A to 16E illustrate exemplary frames 1602A to 1602E of an implant device 110, in accordance with some embodiments. Each of the frames 1602A, 1602B, 1602C, 1602D, and 1602E includes a main frame component 1432 and one or more side frame components 1434, where a respective side frame component 1434 is configured to hold a respective magnet 1404. Details of main frame component 1432, side frame component 1434, and magnet 1404 are described in, for example, FIGS. 14A to 14D and 15A to 15C, and are not repeated for the sake of brevity.

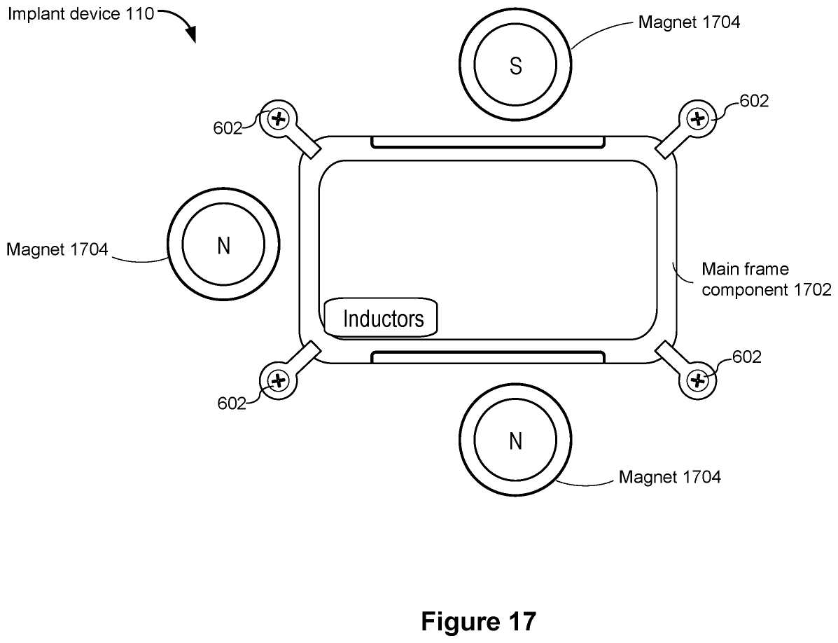

FIG. 17 illustrates an implant device 110, in accordance with some embodiments. The implant device 110 includes a main frame component 1702 and one or more magnets 1704 that are separate from the main frame component 1702. In some embodiments, a respective magnet 1704 is positioned on a respective edge (e.g., along the length or width) of the main frame component 1702. In some embodiments, a respective magnet 1704 is positioned at a respective corner of the main frame component 1702, such as at or near a respective screw 602.

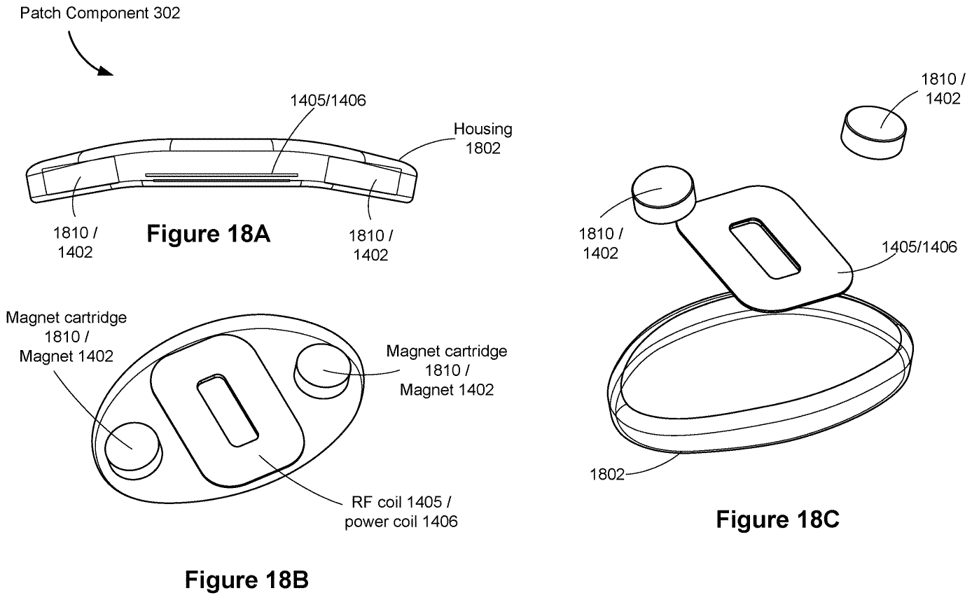

FIGS. 18A to 18C illustrate the patch component 302 (e.g., wearable device 130), in accordance with some embodiments. In some embodiments, the patch component 302 includes a housing 1802 with a shape or contour that is adapted to conform to the curvature of a scalp of a human subject. In some embodiments, the patch component 302 includes one or more magnet cartridges 1810, where a respective magnet cartridge 1810 is configured to hold a respective magnet. A respective magnet cartridge 1810 can be made of a nonmagnetic, biocompatible metal such as titanium, titanium alloy, tantalum, nitinol (nickel-titanium), or stainless steel, or any combination thereof.

Turning to some example embodiments:

In one aspect, a cranial implant device (e.g., implant device 110) includes a frame (e.g., frame 600, 700, 800, 900, 1000, 1100, 1200, 1430, 1602A to 1602E, and 1702) that is configured to be anchored to a skull of a patient (e.g., patient 1300 or human subject 102). In some embodiments, the frame (e.g., frame 600, 700, 800, 900, 1000, 1100, and 1200) includes one or more channels (e.g., slots) (e.g., channel 606, 706, 806, 906, 1016, 1106, or 1206) positioned along one or more sides of the frame. The one or more channels are configured to hold a plurality of first magnets (e.g., magnets 608). The frame includes a mating area (e.g., a recessed area) (e.g., mating area 604, 704, 804, 904, 1014, 1104, or 1204) configured to mate with a hermetically sealed package (e.g., hermetic package 204). The hermetically sealed package configured to be inserted through an opening in the skull and positioned in the mating area. This is illustrated in, for example, FIGS. 6B, 7B, 8B, 8C, 9B, 10B, 11B, and 12B. In some embodiments, the frame includes one or more frame components, such as a main frame component 1432 and one or more side frame components 1434. In some embodiments, a respective frame component includes one or more channels (e.g., slots) positioned along one or more sides of the frame component. In some embodiments, the frame includes a shape, contour, or curvature that is adapted to conform to the curvature of the brain of a human subject.

In some embodiments, adjacent magnets of the plurality of first magnets have opposite polarities. This is illustrated in, for example, FIGS. 6B, 7B, 8B, 8C, 9B, 10B, 11B, and 12B.

In some embodiments, the plurality of first magnets (e.g., magnets 608 or magnets 1404) are configured to magnetically couple (e.g., align) the frame to an external device that is positioned external to the patient. For example, in some embodiments, the external device is a wearable device 130. In some embodiments, the external device is a patch component 302 of a wearable device 130.

In some embodiments, the plurality of first magnets are configured to align the external device to the cranial implant device in multiple axes. For example, the multiple axis can include an x-axis, a y-axis, an x-y-rotation axis, a pitch axis and/or a roll axis.

In some embodiments, the external device includes a plurality of complementary magnets (e.g., complementary magnets 1302 or magnets 1402). The plurality of first magnets (e.g., magnets 608) are configured to magnetically couple to the external device via the plurality of complementary magnets.

In some embodiments, the one or more channels comprise one continuous channel (e.g., channel 606) that spans two adjacent sides of the frame. This is illustrated in FIGS. 6A to 6C.

In some embodiments, the one or more channels comprise two channels (e.g., channel 706-1 and channel 706-2) that are arranged on two adjacent sides of the frame. This is illustrated in FIGS. 7A and 7B.

In some embodiments, the one or more channels comprise one continuous channel (e.g., channel 906) (e.g., a U-shaped channel) that spans three adjacent sides of the frame. This is illustrated in FIGS. 9A and 9B.

In some embodiments, the one or more channels comprise three channels (e.g., channels 1006-1, 1006-2, and 1006-3) that are arranged on three adjacent sides of the frame. This is illustrated in FIGS. 10A and 10B.

In some embodiments, the one or more channels comprise one continuous channel (e.g., channel 1106) that spans four adjacent sides of the frame. This is illustrated in FIGS. 11A and 11B.

In some embodiments, the one or more channels comprise four channels (e.g., channels 1206-1 to 1206-4) that are arranged on four adjacent sides of the frame. This is illustrated in FIGS. 12A and 12B.

In some embodiments, the one or more channels comprise two rectangular channels (e.g., channel 806-1 and channel 506-2) positioned on opposite sides of the frame. This is illustrated in FIGS. 8A, 8B, and 8C.

In some embodiments, the one or more channels comprise one ring-shaped or annular-shape channel (e.g., channel 1106) positioned on all sides of the frame. This is illustrated in FIGS. 11A and 11B.

In some embodiments, the implant device 110 includes a set of electronic components (e.g., implant electronics 206) enclosed in the hermetically sealed package.

In some embodiments, the set of electronic components includes one or more of: a wireless power coil, a wireless communication antenna, and control circuitry.

In some embodiments, the set of electronic components includes a wireless power coil. The plurality of first magnets (e.g., magnets 608) are configured to magnetically align (e.g., orient) the external device (e.g., patch component 302) with the wireless power coil to facilitate wireless electrical powering.

In some embodiments, the set of electronic components includes a wireless communication antenna. The plurality of first magnets (e.g., magnets 608) are configured to magnetically align (e.g., orient) an external device with the wireless communication antenna to facilitate high bandwidth, wireless data communication.

In some embodiments, the set of electronic components is configured to receive electrical power from an external device that is magnetically coupled to the implant device 110 via the plurality of first magnets.

In some embodiments, the set of electronic components is configured to receive electrical power from the external device via inductive coupling or capacitive coupling (e.g., wirelessly).

In some embodiments, the cranial implant device includes an energy storage device (e.g., a battery) that is configured to provide electrical power to at least some of the set of electronic components.

In some embodiments, the cranial implant device does not include a battery.

In some embodiments, the frame and the hermetically sealed package 204 are configured to be located above a dura layer (dura mater) of the skull.

In some embodiments, the implant device includes an electrode array (e.g., electrode array 208) configured to be positioned adjacent to the hermetic package (e.g., at a bottom of the hermetic package, toward an interior of the skull of the patient). The electrode array 208 is configured to acquire neural activity from the patient. In some embodiments, the neural activity comprises sensorimotor cortex activity, which refers to the neural activity within the brain region that integrates sensory information with motor commands, essentially allowing the body to respond to sensory input by initiating movement.

In some embodiments, an edge of the electrode array is configured to align with an edge of the hermetic package.

In some embodiments, the electrode array is configured to be positioned under a dura layer (e.g., dura mater) of the skull.

In some embodiments, the electrode array comprises an array of electrocorticography (ECoG) electrodes. The ECoG electrodes record electrical activity directly from the surface of the brain with high spatial resolution (e.g., at the mm scale or sub-mm scale).

In some embodiments, the implant device further includes a removable cover (e.g., cover 218) piece that is configured to cover the frame. This is illustrated in FIG. 6D.

In some embodiments, the plurality of first magnets are configured to be removable (e.g., replaceable).

In some embodiments, the hermetically sealed package 204 comprises a first hermetically sealed package. The plurality of first magnets (e.g., magnets 608) are enclosed in a second hermetically sealed package (e.g., hermetically sealed package 610, 710, 810, 910, 1010, 1110, or 1210) that is distinct from the first hermetically-sealed package. The second hermetically sealed package is configured to mate with (e.g., fit into) the one or more channels.

In some embodiments, the plurality of first magnets are welded within the second hermetically-sealed package.

In some embodiments, the second hermetically sealed package (e.g., hermetically sealed package 610, 710, 810, 910, 1010, 1110, or 1210) is made of a biocompatible material, such as titanium or silicone.

In another aspect, a system includes a cranial implant device (e.g., plan device 110) and an external device (e.g., patch component 302 or wearable device 130). The external device is configured to provide constant power to the cranial implant device.

In some embodiments, the cranial implant device does not include on-board battery storage.

In some embodiments, the cranial implant device and the external device are magnetically coupled to each other. This is illustrated in FIG. 13.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

Some embodiments may be described based on the following clauses:

Clause 1. A cranial implant device, comprising: (1) a frame that is configured to be anchored to a skull of a patient, the frame including: (a) one or more channels positioned along one or more sides of the frame, the one or more channels configured to hold a plurality of first magnets; and (b) a mating area configured to mate with a hermetically sealed package; and (2) the hermetically sealed package configured to be inserted through an opening in the skull and positioned in the mating area

Clause 2. The cranial implant device of Clause 1, wherein adjacent magnets of the plurality of first magnets have opposite polarities

Clause 3. The cranial implant device of any of Clauses 1-2, wherein the plurality of first magnets are configured to magnetically couple the frame to an external device that is positioned external to the patient.

Clause 4. The cranial implant device of Clause 3, wherein the plurality of first magnets are configured to align the external device to the cranial implant device in multiple axes

Clause 5. The cranial implant device of any of Clauses 3-4, wherein: (i) the external device includes a plurality of complementary magnets; and (ii) the plurality of first magnets are configured to magnetically couple to the external device via the plurality of complementary magnets.

Clause 6. The cranial implant device of any of Clauses 1-5, wherein the one or more channels comprise one continuous channel that spans two adjacent sides of the frame.

Clause 7. The cranial implant device of any of Clauses 1-5, wherein the one or more channels comprise two channels that are arranged on two adjacent sides of the frame.

Clause 8. The cranial implant device of any of Clauses 1-5, wherein the one or more channels comprise one continuous channel that spans three adjacent sides of the frame.

Clause 9. The cranial implant device of any of Clauses 1-5, wherein the one or more channels comprise three channels that are arranged on three adjacent sides of the frame.

Clause 10. The cranial implant device of any of Clauses 1-5, wherein the one or more channels comprise one continuous channel that spans four adjacent sides of the frame.

Clause 11. The cranial implant device of any of Clauses 1-5, wherein the one or more channels comprise four channels that are arranged on four adjacent sides of the frame.

Clause 12. The cranial implant device of any of Clauses 1-5, wherein the one or more channels comprise two rectangular channels positioned on opposite sides of the frame.

Clause 13. The cranial implant device of any of Clauses 1-5, wherein the one or more channels comprise one ring-shaped channel positioned on all sides of the frame.

Clause 14. The cranial implant device of any of Clauses 1-5, wherein the one or more channels comprise at least two channels that are arranged orthogonally.

Clause 15. The cranial implant device of any of Clauses 1-14, further comprising a set of electronic components enclosed in hermetically sealed package.

Clause 16. The cranial implant device of Clause 15, wherein the set of electronic components includes one or more of: a wireless power coil, a wireless communication antenna, and control circuitry.

Clause 17. The cranial implant device of Clauses 15-16, wherein the set of electronic components includes a wireless power coil; and the plurality of first magnets are configured to magnetically align an external device with the wireless power coil to facilitate wireless electrical powering.

Clause 18. The cranial implant device of Clauses 15-17, wherein the set of electronic components includes a wireless communication antenna; and the plurality of first magnets are configured to magnetically align an external device with the wireless communication antenna to facilitate high bandwidth, wireless data communication.

Clause 19. The cranial implant device of any of Clauses 15-18, wherein the set of electronic components is configured to receive electrical power from an external device that is magnetically coupled to the cranial implant device via the plurality of first magnets.

Clause 20. The cranial implant device of Clause 19, wherein the set of electronic components is configured to receive electrical power from the external device via inductive coupling or capacitive coupling.

Clause 21. The cranial implant device of any of Clauses 15-20, further comprising an energy storage device that is configured to provide electrical power to at least some of the set of electronic components.

Clause 22. The cranial implant device of any of Clauses 1-21, wherein the cranial implant device does not include a battery.

Clause 23. The cranial implant device of any of Clauses 1-22, wherein the frame and the hermetically sealed package are configured to be located above a dura layer of the skull.

Clause 24. The cranial implant device of any of Clauses 1-23, further comprising an electrode array configured to be positioned adjacent to the hermetically sealed package, the electrode array configured to acquire neural activity from the patient.

Clause 25. The cranial implant device of Clause 24, wherein an edge of the electrode array is configured to align with an edge of the hermetic package.

Clause 26. The cranial implant device of Clauses 24-25, wherein the electrode array is configured to be positioned under a dura layer of the skull.

Clause 27. The cranial implant device of any of Clauses 24-26, wherein the electrode array comprises an array of electrocorticography (ECoG) electrodes.

Clause 28. The cranial implant device of any of Clauses 1-27, further comprising a removable cover piece that is configured to cover the frame.

Clause 29. The cranial implant device of any of Clauses 1-28, wherein the plurality of first magnets are configured to be removable.

Clause 30. The cranial implant device of any of Clauses 1-29, wherein the hermetically sealed package comprises a first hermetically sealed package; the plurality of first magnets are enclosed in a second hermetically sealed package that is distinct from the first hermetically sealed package; and the second hermetically sealed package is configured to mate with the one or more channels.

Clause 31. The cranial implant device of Clause 30, wherein the plurality of first magnets are welded within the second hermetically sealed package.

Clause 32. The cranial implant device of any of Clauses 30-31, wherein the second hermetically sealed package comprises a biocompatible material.

Clause 33. A system, comprising: (i) a cranial implant device; and (ii) an external device, the external device configured to provide constant power to the cranial implant device.

Clause 34. The system of Clause 33, wherein the cranial implant device comprises the cranial implant device of any of Clauses 1-32.

Clause 35. The system of any of Clauses 33-34, wherein the cranial implant device does not include on-board battery storage.

Clause 36. The system of any of Clauses 33-35, wherein the cranial implant device and the external device are magnetically coupled to each other.

It will be understood that, although the terms “first,” “second,” etc. may be used herein to describe various elements, these elements should not be limited by these terms. These terms are only used to distinguish one element from another. The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or embodiments.

As used herein, the term “and/or” encompasses any combination of listed elements. For example, “A, B, and/or C” entails each of the following possibilities: A only, B only, C only, A and B without C, A and C without B, B and C without A, and a combination of A, B, and C.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A cranial implant device, comprising:
a frame that is configured to be anchored to a skull of a patient, the frame including:
one or more channels positioned along one or more sides of the frame, the one or more channels configured to hold a plurality of first magnets; and
a mating area configured to mate with a hermetically sealed package; and
the hermetically sealed package configured to be inserted through an opening in the skull and positioned in the mating area.

2. The cranial implant device of claim 1, wherein adjacent magnets of the plurality of first magnets have opposite polarities.

3. The cranial implant device of claim 1, wherein the plurality of first magnets are configured to magnetically couple the frame to an external device that is positioned external to the patient.

4. The cranial implant device of claim 3, wherein the plurality of first magnets are configured to align the external device to the cranial implant device in multiple axes.

5. The cranial implant device of claim 1, wherein the one or more channels comprise one of:
one continuous channel that spans two adjacent sides of the frame;
two channels that are arranged on two adjacent sides of the frame;
one continuous channel that spans three adjacent sides of the frame;
three channels that are arranged on three adjacent sides of the frame;
two rectangular channels positioned on opposite sides of the frame; and
one ring-shaped channel positioned on all sides of the frame.

6. The cranial implant device of claim 1, further comprising:
a set of electronic components enclosed in hermetically sealed package, the set of electronic components including one or more of: a wireless power coil, a wireless communication antenna, and control circuitry.

7. The cranial implant device of claim 6, wherein:
the set of electronic components includes a wireless power coil; and
the plurality of first magnets are configured to magnetically align an external device with the wireless power coil to facilitate wireless electrical powering.

8. The cranial implant device of claim 6, wherein:
the set of electronic components includes a wireless communication antenna; and
the plurality of first magnets are configured to magnetically align an external device with the wireless communication antenna to facilitate high bandwidth, wireless data communication.

9. The cranial implant device of claim 6, wherein:
the set of electronic components is configured to receive electrical power from an external device that is magnetically coupled to the cranial implant device via the plurality of first magnets; and
the set of electronic components is configured to receive electrical power from an external device via inductive coupling or capacitive coupling.

10. The cranial implant device of claim 1, wherein the cranial implant device does not include a battery.

11. The cranial implant device of claim 1, further comprising a removable cover piece that is configured to cover the frame.

12. The cranial implant device of claim 1, wherein the plurality of first magnets are configured to be removable.

13. The cranial implant device of claim 1, wherein:
the hermetically sealed package comprises a first hermetically sealed package;
the plurality of first magnets are enclosed in a second hermetically sealed package that is distinct from the first hermetically sealed package; and
the second hermetically sealed package is configured to mate with the one or more channels.

14. The cranial implant device of claim 13, wherein the plurality of first magnets are welded within the second hermetically sealed package.

15. The cranial implant device of claim 13, wherein the second hermetically sealed package comprises a biocompatible material.

16. A system, comprising:
a cranial implant device, the cranial implant device including:
a frame that is configured to be anchored to a skull of a patient, the frame including one or more channels positioned along one or more sides of the frame, the one or more channels configured to hold a plurality of first magnets;
a hermetically sealed package, the hermetically sealed package configured to be inserted through an opening in the skull;
a set of electronic components enclosed in hermetically sealed package, the set of electronic components including one or more of: a wireless power coil and a wireless communication antenna;
an external device configured to provide constant power to the cranial implant device; and
the plurality of first magnets configured to magnetically couple to the external device via a plurality of complementary magnets.

17. The system of claim 16, wherein:
the set of electronic components includes a wireless power coil; and
the plurality of first magnets are configured to magnetically align an external device with the wireless power coil to facilitate wireless electrical powering.

18. The system of claim 16, wherein the cranial implant device does not include on-board battery storage.

19. The system of claim 16, wherein:
the set of electronic components includes a wireless communication antenna; and
the plurality of first magnets are configured to magnetically align the external device with the wireless communication antenna to facilitate high bandwidth, wireless data communication.
